SEQUENCE LISTING (25 sequences)

SEQ ID NO: 1 - DNA, 861 bp, Medicago truncatula
  source 1..861: mol_type = other DNA
atggctctctccaacttaaaatctaacagaactctttcttcatcactcatcacaatcttcatcatttccctcttcctacaataccacaatatcaaatcacaatcatcatggcaatcacgacaagtaccacgatcagaaaccgttgctttcagcataacggaattcgagaaagaaaatcccgacatatttttaaggggtgacacctcaatctccgacggcattttaagactaacaaaaactgaccaatctggtaaaccactccctaataccgttggacgtgcgacatacctcacgccaattcatatttgggataaaactagtggagaactagctgatttctccactagtttctcctttatcgtgaacacaaacgattcagatctccatggagacggattcgctttctatctgggaccactacattttgacgtaccaaaaaattcaagcggagggtatcttggtcttttcgacccagaaaatgcatttcctccatccaaaaccccaatattagctattgaatttgatggttttacaaatgaatgggaccctccttcatcatttcaatctcctcacattggaattgatgttgggtcaattgtttcccttgaatatgcacaatggcctattaattttgtgccaagaaatgctttaggagaagctaacataaactataactcagagtctaaaagattgagtgtgtttgtggcttatcctgggactcagtggaattccactagggtttcagttgttgttgatctaaggagtgttttgcctgaatgggttagaataggtttctctgcaactacaggagaactggttgaaacacatgatatcattaattggtcttttgaatcagccttg

SEQ ID NO: 2 - protein, 287 aa, Medicago truncatula
MALSNLKSNRTLSSSLITIFIISLFLQYHNIKSQSSWQSRQVPRSETVAFSITEFEKENPDIFLRGDTSISDGILRLTKTDQSGKPLPNTVGRATYLTPIHIWDKTSGELADFSTSFSFIVNTNDSDLHGDGFAFYLGPLHFDVPKNSSGGYLGLFDPENAFPPSKTPILAIEFDGFTNEWDPPSSFQSPHIGIDVGSIVSLEYAQWPINFVPRNALGEANINYNSESKRLSVFVAYPGTQWNSTRVSVVVDLRSVLPEWVRIGFSATTGELVETHDIINWSFESAL

SEQ ID NO: 3 - DNA, 2000 bp, Medicago truncatula
  source 1..2000: mol_type = other DNA
actaggatagttgcaaccaatccaacactcaaaccacttaaaggactttaaatttattatccatttaaatttattatccataataatttcattcgagaaagaatgattattagtactagatagataaagaacaattcaatattactaatttatgatatttgggtttggtctaaaataagttttagtttcatccatggtgcgtaattgtttgaattgttgaatagcatagattaagtaattgcagcaagagttcaataggtttgaccaaatatagaaaacaaacgatatagggatgcttgtggggaagttattactccttgagttcaccatcctatggaagcctaaaagtgtgtgagaaaattccaaagttgagtattgttgattgttaattgaatcatcatagttaacatgttgaattgtttctaatgtgtttcatcatattgttcaagtaggattaggatcattgccacttctcctgtataagttgtttttgatggctctcctacataacataaccagcgaataactttgcttgcctgcaatctaaatgtatctttttctattttctattaacataattgtcttgtgttctgaatcttctagctttgcttataaaatgttgaataccataaacatatgtagctgttttgctcatttgcataccttctgaaagtattttaagttgagacttcataaaaatattgcattgagttttagggaagaaaaattctaaaaatgtgagtgtccatactcctatattattgtctttttgagatgaaggacaccactatatcattgagttataactaaaatgaaggttcaaaatatagttcttacacatccataaatgagttaataaaaataagaaggaaacaactcaatttatgatgtttgaaaaaaaaaagaatgttggagtactacgtccacaatccatactcctaaatcaacaatcttttcaactcttctgattttgaaattactacacaaataacatggatggggaagagatcaaacacaactatatatgtgtgttccagacccatacctgataatcgtgcacaattatacaagctaaagttttaatactgctcagtaatctaaagtccctcaaaaaataagtattgtaattgaagatcagaatataacaaaattgtgtgatgcaacttcaaatactattcaaaaggaaggaagtctttgtagcaattattttttaaggggtgactgattttttactctttaaaattcgattgtctttagatctattcgatgtcgctaatgtattttcacgggtcgattttagctatttgagataattagtggtgtatctaaatagatagacctgactactatctcgaatattgatcgtagtatatgctcttttgatgctttgataaatattttaaaaaggtgcacatttctaatgtttgtgtaatatgtttacttaattctcacatttataatcaacaacaatcttttagaaaaatgcaactatttcccttaatttgattatttttccttcaaaacaatttttttacaaaatacgttattcaacctgtgcgaagcacgggttttaactagttataataataatcagacgatttctcataaatgatcaccacatagtacatcgacttgctggtaataggtatttgttgtaaaatgataaactttcagtaatttgtaaacatattttcaccaataaataagcactccacaattatacccccttctcctcgtattccatgatcgttttgaaaagaaacatgaaaatttcagtgttttttaacaacattttttggacttaatatctagctttttttccttggattttccacctaaattagttggcaaatggcaacacacctctatgcaagttgcttgtcgtattcattcggcaattgattactatatatactttataaactattttcctagccggccaacataacaccctcctataaaataaaaacacattttcccttagtttcaagcatcacaaa

SEQ ID NO: 4 - protein, 286 aa, Medicago truncatula
MALSNLKPNRSTLSSALIKIYITFVFLQYHVNSQYSPSPSHQPKNTVSFTILEFEKENPDIFLRGDASISGGILRLTKTDQSGEPIQKSVGRATHLTPIHIWDKTSGKLADFSTRFFFFVNTNGSELHGDGFAFYIGPLHFEVPKNSSGGYLGLFDPETAFDPSKNPIIAIEFDSFTNGWDPASPSQYPHIGIDVGSIDSVATVDWPVDFLPRNALGEANINYNSESKRLSVFVNYPGSGRKATGVSFVVDLRSVLPEWVRVGFSAATGELVEIHDIINWSFESSL

SEQ ID NO: 5 - protein, 279 aa, Medicago truncatula
MALSSALIKIFITFLFLQNHVNSQYSSPSKPQPSLGSTISFSMTKFDDESPNIFVKGDASISNGVLSLTKTDKYSGKPLQKSVGRATHLTPIHIWDETSGELADFSTSFSFIVNTNGSRLHGDGFTFFLGPLHFDLPKNSSGGYLGLFNPETALIPSQNPIVAIEFDSFTNGWDPASPSQYPHIGIDVGSIDSRATVNWPLDFVQTNALGEASINYNSESKRLSVFVAYPGSGKNATGVSFVVDLRSVLPEWVRVGFSAATGELVETHDIINWSFEAAL

SEQ ID NO: 6 - protein, 278 aa, Medicago truncatula
MTLSSALIKIFITFLFLQNHVNSQYSSPSKPQPSLGSTISFSITKFDDESPNIFVKGDTSISNGVLSLTKTDKYSGKPLQKSVGHATHLTPIHIWDETSGELADFSTSFSFIVNTNGSSLHGDGFTFFLGPLHFDLPKNSSGGYLGLFNPETALIASQNPIVAIEFDSFTNGWDPASPSQYTHIGIDVGSIDSVSTADWPLNVLPRNALGEARINYNSESKRLSAFVDYPGLGESTGVSFVVDLRSVLPEWVRVGFSAATGELVETHDIINWSFEAAL

SEQ ID NO: 7 - protein, 273 aa, Myrciaria dubia
MAFSNSITFLFITFLFLKYPNVNSQSQVSGLETFTFTINQFKRANPNIVFEGDASVSGGILKLTKTDQLGKPLSKSVGRAIHLTPIHIWDKINGELADFSTTFIFAVNTNGSQLHGDGFTFFLGPIDFDLPKNSSGGYLGLFNPETALIPSENPILAIEFDSFTNEWDPTAPFQSPHIGIDVGSIDSVATTDWPIDFVQRNAIGEASINYNSESKRLSVFVAYPGSGRNVTGVSSIVDLRSVLPEWVRVGFTAATGELVETHDIISWSFESAL

SEQ ID NO: 8 - protein, 288 aa, Glycine max
MAFPNSKPNLLQSLSPLIKFFIPFLLLLQHHSVKSQQQPPSPMSAYETVGIDFSFFDKDDPNVLLIGNASVSGGALRLTNTDQLGKPVPHSVGRVVHITPIHLWNKNNGHLADFTSDFSFVVNPKGSALRGDGFAFFLTSANLNFLIPKNSSGGYLGLFKPETALDPSKNQIVAIEFDSFTNDWDPNSPNQSPHVGIDVDSIKSVATVPWPSELEPDNAVAHASLNYNSEDKRLSVFVGYPDNRNATVSAIVDLRNVLPEWISVGFSASTGDLVETHDILNWSFEAAL

SEQ ID NO: 9 - protein, 288 aa, Cajanus cajan
MAFLNSSKPNLLQSLSPLSTLLIKFLIPFLLLQHHIVKSQFSPALANDESIFFGISLFSKEDPNIILLGNASISGGVLRLTNNDQFGQPVTHSVGRAVHLTPIHLWNRHNGDLADFGAAFSFVVNSKGSTLHGDGFAFFLAPADFHFPKNSSGGYLGLFNPDNAFDPFRNQVVAIEFDSFTNEWDPSSPVQVPHIGIDVGSVKSVAAVPWPSELESPSAIAHASLNYNSESKRLSVFVGYPDNRNATVSALVDLRNILPEWIRVGFSASTGELVETHDILSWSFEATL

SEQ ID NO: 10 - protein, 283 aa, Glycine max
MAFSNSKPNLLQSLSPLIKFLIPFLLLQHSVNSQQPPSPMSAYETVGFGFGFFDKDDPNVFLLGNASVSGGALRLTNTDQLGKPVPHSVGRALHVTPIHLWNKNNGELADFSSGFSFVVNPKGSTLRGDGFAFFLAPANLNFPKNSSGGYLGLFNPETALDPSKNQIVAIEFDSFTNDWDPNSPNQSPHVGIDVDSIKSVATVPWPSELEPDNAVAHASLNYNSESKSLSVFVGYPDNRNATVSTIVDLRNVLPEWIRVGFSASTGDLVETHDILNWSFEAAL

SEQ ID NO: 11 - protein, 283 aa, Trifolium subterraneum
MVFSNSKFNTVQSRVLSLPIFITFLLLQYHNVESQTPLTRSENVAFSITQFEEENTDIFTRGDATISRGILRLTKTDQYGKPLPNSVGRATYVTPIHIWDKASGKLADFSTGFSFIVNTNDEPLHGDGFAFFIGPIHFDLPKNSSGGYLGLFNPETAHVPSKNPIVAIEFDGFTNVWDPPSSYQSPHIGIDVGSIDSVAYTQWPINFVQKNPIGEASINYNSESKSLSVFVAYPGTEWNSTSVSFVVDLRSVLPEWVRVGFSAATGQLVETHEIINWSFESSL

SEQ ID NO: 12 - protein, 289 aa, Trifolium subterraneum
MALSNLKSNRTTLSSQLIKIFIAFLFLQYHNVNSQYSPSPSQELARTETFSFTVRQFDEENPNIFLGGGASTTGGILKLTKADQNGKPLQNSVGRATYFTLIHIWDKASGELADFSVGFSFVVNTNGSRLHGDGFTFFIGPVHFEIPKNSSGGYLGLFNPETAHIPAQNPIIAIEFDSFTNGWDPATPSQFPHIGIDVGSIDSVATGNWPIDFVQANPLGEASINYNSESKKLSVFVAYPGSEREGTSVSCVVDLRSVLPEWVRVGFTASTGELVETHDIINWSFESAL

SEQ ID NO: 13 - DNA, 618 bp, Medicago truncatula
  source 1..618: mol_type = other DNA
atggaagaatcgaaaaacaaacaattagagttagttgacaccctaactccactacctcaatctgaatctgaacctcgagagttttcctacttccttgaagaaaaggaaccggggaacgaaggaacttcaagcagtgttgttaaacaagagcgtgtcgtgagtgatcatgctacttcttcagttgatcaaactactgctgctggaacagatacaaaggactcagttgaccgagatgctgtactagcaagggttgaatcgcagaaaagattggctttaattaaagcatgggaagaaaatgagaagacaaaagtagagaatagggcatataagatgcaatctgctgttgatttgtgggaggatgataagaaagcatcaattgaagcaaaattcaaaggaattgaggtaaaattggacagaaagaagagtgaatatgttgaagtaatgcaaaacaaaataggtgaaattcataaatcagcagaagagaaaaaggcaatgatagaagctcaaaaaggggaagaaatcctcaaggtagaggagacagctgcaaagtttcgtactcgtggctatcagccaagaagacttcttggatgtttcagcggtttaaggtttttcagttag

SEQ ID NO: 14 - protein, 205 aa, Medicago truncatula
MEESKNKQLELVDTLTPLPQSESEPREFSYFLEEKEPGNEGTSSSVVKQERVVSDHATSSVDQTTAAGTDTKDSVDRDAVLARVESQKRLALIKAWEENEKTKVENRAYKMQSAVDLWEDDKKASIEAKFKGIEVKLDRKKSEYVEVMQNKIGEIHKSAEEKKAMIEAQKGEEILKVEETAAKFRTRGYQPRRLLGCFSGLRFFS

SEQ ID NO: 15 - DNA, 2000 bp, Medicago truncatula
  source 1..2000: mol_type = other DNA
tgaatgtgatattttttagggttaatagtgcttttcacctctgtaatatatgtcatttccagttttcgccctataaaattttcagtttgatttgcaccctcgtaaaatttttattttctagaaaacacccttaataggccattcaaaagccaaaatttcttgaaaaaaaaaaactgaaaatgacctactagggatgttttctggattttttttttacaagggtgcaaatcaaaccaaaaactttatagaggcgaaaatcgaaaatgacatatattacaggggtgaaaaacattattaaccctattttttatgtataaccgtagagattaatctcttaaattgaatagaaccatggaaagattattcctcaaagatttaacaccatttacaattggccaattggtattataaattcgttaactaccactattttattcattgtatcattatctaatttatctcaaatatactcccccgtcacttgttttgagcaaaaaaaagtcattttttatcctaaattataagtaaaaaagattaacttttaccctatttaatcacgttgtttcaaaaactcattttttccaaataaaaaaacttttcttattcacatgaaattgaattaaatttgttttcccttccattttctccataaccaataaaaattatttttacatcttctgttaaaacttattcttagaaaaacacacaaaattatactccaaattgttatattttagtttttttaataaatgtgaattgttttttttccttattacttgggagagagggagtacgtgttcatcttaaataaactactccatcaataagagaatatagtagcttatgttaaaaaaattatacgctactccttcaatatctcaagtgagaagagtgtaaattttaaataaaggggaagaggaactttggccctccttaacaccaaagaaaggacaagacggagggaataacaatttaccctattttaaaagaaagtgatggtttttttattaaaaaaaacctagtattttcaaagaataaacatcaatattctcttgaaaaaataatcatcttcatcattgttaattttagttttcaatcttaatttatacatttaggaacacatctcctttattttataatgttcttttttttagaagtatcttacaatccctaaaataaaagaagtattttacaatgttttaaatattaaactaataaacttttttttgaggctattaaattaataaacttaaaaaatgattcaaatagaatttatattatacgataaagattgaactaaattaaatcaaatctaaatttcataattttaaatcaatataaaattctaattttaaattacgttagtttatataaggggttaaattgaatgatattcttgaccaatttttattttgaatgacattttcataagtcttagccaaaacttcaaacgccttgcttagatttagatcacctctattttggtttccgaaattgttaggccggatgccatgatcggggttcgaaccccggtaccttcacttgtgtgtgtgagtttataatggctttgccatttcgtctatctaccaaaaaaaaaaaaaattaaaaaatgtaagttgtgtaaaatgaataccttaaaaaaaattatttcatttcgtgaccaacatctcaatggaactgaataccttactttggttggcatacaagcagtgtaaaatgtaagttgtgaaatgaaccccaagtatagaaaatatatggtaaaaatatttgaaataattaataaaagacaaagcagtgaatatccgcataactgtctattagtggaccccgtttgagtcttcacaatgtctcttacataaacctttgaggccattggagtggtgaccaaacagcttggcttgctgtaagcaaaaacaacaatttcatctctatttaagtgtgatcacaaacatattctaagtttaatagaaattctaatgctgtaaccctagaaatacatt

SEQ ID NO: 16 - protein, 207 aa, Cicer arietinum
MEESSQSESVATPITPPPQSPPQLRELSHFVKEKEPENQGTSTNIVKQEYTQDFFQPKDHATSSLDQIPDAGTDTKDSVDRDTVLARVESQKRLALIKAWEENEKTKVENRAFKMQSAVDLWEDNKKASIEAKFKGIEVRLDKKKTEYVEIMQNKIAEIHHSAEEKKAMIEAQKGEEILKVEETAAKFRTRGYVPRRLLGCFGLKLF

SEQ ID NO: 17 - protein, 203 aa, Trifolium pratense
MEETLKNEQSIDTPNAPPDQPLELSYFVEEKKPKNEGTSSSIVKQERIVRDHATSPLNQIPPAAGTDTKDSVDRDAVLARVESQKRLALIKAWEENEKTKVENRAYKMQSAVDLWEDNKKSSIEAKFKGIEVKLDKKKSEYIEVMQNKIAEIHLSAEEKKAMIEAQKGEEIVKVEETAAKFRTRGYEPRRLLGCFGFGVRAFR

SEQ ID NO: 18 - protein, 207 aa, Lotus japonicas
MGEEETKHCDQCGASASASASEATVLSQLRLSEVLKLKESQNAESSNSTLTITRQGSTTNPSYPLDQDLDAETDNINTSIDRDAVLARVESQKRLALIKAWEENEKTKVDNKAYKLQCAVDMWEKTKKASTQAKIKKIEENMDRKKADYVEIMQNKIAETHRLADEKKALIEAQKGEEVLKVEETAAKFRTRGYVPKKFLSCFNFSF

SEQ ID NO: 19 - protein, 194 aa, Glycine max
MGVSGNQRGKEVENTHTSSGVRQEYAFSPLNLSLFAKWNRFRSFLVILSKKVKKKVPTTTRTDSKDSVDRDAVLARVESEKRLALIRAWEESEKTKAENRAYKRHNAVVLWENSKKASAEAHLKRIEEKLDRNKAKCVEKMQNNVAEIHRTAEEKRAMIEANRGEEFLEIEEKAAKFRTRGYSPRKYLPCFGSS

SEQ ID NO: 20 - DNA, 1944 bp, Medicago truncatula
  source 1..1944: mol_type = other DNA
gagacatcagcatatatttttaatatcaaaccaaaagatatttgatgtccaaagaagagataaatataaagttagaagtatgacaataagtcttggttgttatttgccataagaaactctcttttctcttcccctcataatttgcatttcctcacaacaatgtctgccttctttcttccttctagttctcatgctctttttcttgtcctcatgttgttttttctcactaacatctcagctcaaccactatatataagtgaaacaaactttacatgccctgtggattctcctccttcatgtgaaacctatgtagcatacagagctcagtctccaaattttttgagcctatctaacatatctgatatttttaatttgagtcctttacgcattgcaaaagccagtaacatagaagccgaggataagaagctgattccagatcaactgttactagtacctgtaacttgtggttgcactaaaaatcactctttcgcgaatatcacctactcaatcaagcaaggtgacaacttcttcatactttcaatcacttcataccagaatctcaccaattatcttgaatttaaaaatttcaaccccaatctaagtccaactctattgccactagacaccaaagtttcagtccctttattctgcaagtgcccttcaaagaatcaattgaacaaaggaataaagtatcttattacttatgtgtggcaggataatgacaatgttacccttgttagttcaaagtttggtgcatcacaagttgaaatgttagctgaaaacaatcataacttcactgcttcaaccaaccgttcagttttgatccctgtgacaagtttaccaaaacttgatcaaccatcttcaaatggaagaaaaagcagcagtcaaaatctggctcttataattggtatcagcctaggaagtgcttttttcattttagttttaacactatcacttgtgtacgtatattgtctcaaaatgaagagattgaatagaagtacttcatcgtccgagactgcagataagttactttcaggtgtttcaggttatgtaagcaagccaacaatgtatgaaattgatgcaatcatggaaggtacaacgaatctgagtgacaattgtaagattggtgaatcagtttacaaggctaatatagatggtagagttttagcagtgaaaaaaatcaaaaaagatgcttcagaggagctgaaaattctgcagaaggtaaaccatggaaatcttgtgaaactaatgggtgtgtcctctgacaatgatggaaactgttttctggtctatgagtatgctgaaaatggatcacttgaagagtggttgttctcggaatcttcgaaaacttcaaactcggtggtttcgcttacatggtcccagagaataaccattgccatggatgttgcaatcggtctgcaatacatgcatgaacatacttatccacgaataatccacagagacatcacaacaagtaatatccttcttggctcaaattttaaggccaagatagcgaatttcgggatggctagaacttcaaccaactcaatgatgccgaaaattgatgttttcgcttttggggtggttctaatagagttgttaactggcaagaaagctatgacaactaaagaaaatggtgaggttgttattctgtggaaggatttttggaagatttttgatcttgaagggaatagagaagagaggttaagaaaatggatggatcctaagttagagagtttttatcctatagataatgctctaagtttggcttctttggcagtaaattgcactgctgataaatcattgtctagaccaaccattgcagaaattgttctttgtctttctcttctcaaccaaccatcatctgaaccaatgttagaaagatccttgacatctggtttagatgctgaagctactcatgttgttacttctgtaatagctcgt

SEQ ID NO: 21 - protein, 595 aa, Medicago truncatula
MSAFFLPSSSHALFLVLMLFFLTNISAQPLYISETNFTCPVDSPPSCETYVAYRAQSPNFLSLSNISDIFNLSPLRIAKASNIEAEDKKLIPDQLLLVPVTCGCTKNHSFANITYSIKQGDNFFILSITSYQNLTNYLEFKNFNPNLSPTLLPLDTKVSVPLFCKCPSKNQLNKGIKYLITYVWQDNDNVTLVSSKFGASQVEMLAENNHNFTASTNRSVLIPVTSLPKLDQPSSNGRKSSSQNLALIIGISLGSAFFILVLTLSLVYVYCLKMKRLNRSTSSSETADKLLSGVSGYVSKPTMYEIDAIMEGTTNLSDNCKIGESVYKANIDGRVLAVKKIKKDASEELKILQKVNHGNLVKLMGVSSDNDGNCFLVYEYAENGSLEEWLFSESSKTSNSVVSLTWSQRITIAMDVAIGLQYMHEHTYPRIIHRDITTSNILLGSNFKAKIANFGMARTSTNSMMPKIDVFAFGVVLIELLTGKKAMTTKENGEVVILWKDFWKIFDLEGNREERLRKWMDPKLESFYPIDNALSLASLAVNCTADKSLSRPTIAEIVLCLSLLNQPSSEPMLERSLTSGLDAEATHVVTSVIAR

SEQ ID NO: 22 - DNA, 4124 bp, Medicago truncatula
  source 1..4124: mol_type = other DNA
atgaatctcaaaaatggattactattgttcattctgtttctggattgtgtttttttcaaagttgaatccaaatgtgtaaaagggtgtgatgtagctttagcttcctactatattataccatcaattcaactcagaaatatatcaaactttatgcaatcaaagattgttcttaccaattcctttgatgttataatgagctacaatagagacgtagtattcgataaatctggtcttatttcctatactagaatcaacgttccgttcccatgtgaatgtattggaggtgaatttctaggacatgtgtttgaatatacaacaaaagaaggagacgattatgatttaattgcaaatacttattacgcaagtttgacaactgttgagttattgaaaaagttcaacagctatgatccaaatcatatacctgttaaggctaagattaatgtcactgtaatttgttcatgtgggaatagccagatttcaaaagattatggcttgtttgttacctatccactcaggtctgatgatactcttgcgaaaattgcgaccaaagctggtcttgatgaagggttgatacaaaatttcaatcaagatgccaatttcagcataggaagtgggatagtgttcattccaggaagaggtatgtattttctcattttctgccaactgtggttggcacagatggtttgaacttctgtcacatccgttgtaactttgataagtctgaaattccgcagtttgtagattactggtaaattccattataaatgtttaatgtgatttggtgattcttatcaaaagtacttgtataagtatgcgagttagataaaaaaaattatgaccatcttgttctcgtggaaatggactctgataattcataaagtctagccagtgattgtaacaaccaggctttgaacttggtacttccaatcaacttgaccttcaccagacctcattgaccacttgagtcgaaccctttaatttcagttagagtatatttaaatgctaagttactctattatttttcaaagtatatacatggtataaattttgaagttttatgtagttattgtttactttgcagatcaaaatggacatttttttcctttgtattctaggtaagtaacattgattatctcaattttcatttttgaatgatttatagaagaagtaaatattgcttcatataatttggttatatttttctaactttcattttctttttatttttccattcttgcagaacaggtttgtcttttgctattaagatgattatttgttagcttgttcacaaaaatatgagaatggacaaaaggtcaatgcttcctgtgagcttaaatttggttcaatataagcaggtattgctaagggttcagctgttggtatagctatggcaggaatatttggacttctattatttgttatctatatatatgccaaatacttccaaaagaaggaagaagagaaaactaaacttccacaaacttctagggcattttcaactcaagatggtaatatttttaaacattcatattctaagttcttattaaaaatatttcttttaacctatcttatgatataagtatttatttcagtatttgagagagcttgcgaaaatagcttataacatgtttgtttcattaaactgtatttatttcattaaatagtttatacttgctgatttttgtttatgttattggtgaagcctcaggtagtgcagaatatgaaacttcaggatccagtgggcatgctactggtagtgctgccggccttacaggcattatggtggcaaagtcgacagagtttacgtatcaagaattagccaaggcgacaaataatttcagcttggataataaaattggtcaaggtggatttggagctgtctattatgcagaacttagaggcgaggtacgaaactacatgaatttgtttaatagagtgtactttgattttagttttgaacaagttctataaaatattttcaaaaaacttttattttttgtcataacttggaaagaaagtaaagccatttttttttccttcacgttttcattgatttcctctcatgcaacttattgtatgcagaaaacagcaattaagaagatggatgtacaagcatcgtccgaatttctctgtgagttgaaggtcttaacacatgttcatcacttgaatctggtataccatccttttaaaaatcttaagccatatataatatatttaggagatataatcatttatttttatatatggtttgaagaatcatcgtttaactacaaagcaaataaccagtgttagttttgagaacataagaactctataactatcaagcaaaacataatctgtagtagctgtttacaattatctgtcctacacagttagcgaataatttgaaacacactgcagaacattatttgtatgtacttcttgattttgtacatgtttgtatactttttgtataatcagttttgtatttgttctagatattactctgaatttgcctaaattttatgaacaatgtaggtgcggttgattggatattgcgttgaagggtcacttttcctcgtatatgaacatattgacaatggaaacttgggtcaatatttacatggtataggtaagattaacaaaaatgtgctaatatttttatgtgattttacaatattgtcaaacagtcattaatgatggttagatgatttcaggtacagaaccattaccatggtctagtagagtgcagattgctctagattcagccagaggcctagaatacattcatgaacacactgtacctgtttatatccatcgcgacgtaaaatcagcaaatatattgatagacaaaaatttgcgtggaaaggttgcaatttgaccaatcttaatgatctatattataaattttaatttatcacttcttcttttacattaattaactctatgaatggttttgaattcaggttgctgattttggcttgaccaaacttattgaagttggaaactcgacacttcacactcgtcttgtgggaacatttggatacatgccaccagagtatgattcgtttgtattaaattttgagtttaatattagtacaaaaagtacaacaaaaattcagtgattcattcacatttcacaatacatatgtcactttgttatattataaaatgggatatgaccagatgattgtacaattttttttataacaaatgatatttgtataacccttttagtatgtccatggattataaactatcttcaactttcttaattgtagaaaacatgtttgtttattagctgttttttttctctgttgcagatatgctcaatatggcgatgtttctccaaaaatagatgtatatgcttttggcgttgttctttatgaacttattactgcaaagaatgctgtcctgaaaacaggtgaatctgttgcagaatcaaagggtcttgtacaattggtaggtctagataccatatttattaagaaaacactcatttcatgtatatttttagtaaaatatttttaagttagtaattatgtacattttaaattcagtaaactgaatgcattcacttaaaccagaacaaaagttatccttgattattttgtattgcagtttgaagaagcacttcatcgaatggatcctttagaaggtctacgaaaattggtggatcctaggcttaaagaaaactatcccattgattctgttctcaaggtgggaagcattttttcttagcaaaaaattgaatgttatttctttttcttctcaatttgcattatataccaacaaaaaaaaaatgcatatttatgtggtatagcctttcaaatcattgtagtacataagcaaagttcatgttattaaaatataattaaatgtatgcaaaagtgtatagtttgtaaagttactaaactcatttgttttagcactagattttgtcattgaacataacttaagatatgtgaatatttgaattgcagatggctcaacttgggagagcatgtacgagagacaatccgctactacgcccaagcatgagatctatagttgttgctcttatgacactttcatcaccaactgaagattgtgatgatgactcttcatatgaaaatcaatctctcataaatctgttgtcaactaga

SEQ ID NO: 23 - protein, 620 aa, Medicago truncatula
MNLKNGLLLFILFLDCVFFKVESKCVKGCDVALASYYIIPSIQLRNISNFMQSKIVLTNSFDVIMSYNRDVVFDKSGLISYTRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGRDQNGHFFPLYSRTGIAKGSAVGIAMAGIFGLLLFVIYIYAKYFQKKEEEKTKLPQTSRAFSTQDASGSAEYETSGSSGHATGSAAGLTGIMVAKSTEFTYQELAKATNNFSLDNKIGQGGFGAVYYAELRGEKTAIKKMDVQASSEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVLYELITAKNAVLKTGESVAESKGLVQLFEEALHRMDPLEGLRKLVDPRLKENYPIDSVLKMAQLGRACTRDNPLLRPSMRSIVVALMTLSSPTEDCDDDSSYENQSLINLLSTR

SEQ ID NO: 24 - DNA, 372 bp, Arabidopsis thaliana
  source 1..372: mol_type = other DNA
atggagccaaatattccgatccaaagaggagatgagcagtcaaaagttattaaggcatggaaggaactaaagataacaaaggtcaataacaagactcagaagaagctactagatatttcaggatgggagaaaaagaaaactacaaagatcgaatctgaactcgctagaattcagcggaagatggacagtaagaagatggagaaatctgagaaactaaggaacgaaaaagcggcagttcatgcaaaggcacaaaagaagaaggcagatgttcaaaccagacgggctcaagagatccttgatgcggaagaagctgctgctaggtttcaagccgcaggaaagatacccaagaagtcatctttgagctgcttctga

SEQ ID NO: 25 - protein, 123 aa, Arabidopsis thaliana
MEPNIPIQRGDEQSKVIKAWKELKITKVNNKTQKKLLDISGWEKKKTTKIESELARIQRKMDSKKMEKSEKLRNEKAAVHAKAQKKKADVQTRRAQEILDAEEAAARFQAAGKIPKKSSLSCF

(19) United States
(12) Patent Application Publication
(10) Pub. No.: US 2023/0086871 A1
(43) Pub. Date: Mar. 23, 2023

(54) PROTEINS FOR REGULATION OF SYMBIOTIC INFECTION AND ASSOCIATED REGULATORY ELEMENTS

(71) Applicant: University of Freiburg, Freiburg (DE)

(72) Inventors: Thomas Ott, Merzhausen (DE); Lina Siukstaite, Freiburg (DE); Chao Su, Freiburg (DE); Winfried Römer, Bad Krozingen (DE)

(21) Appl. No.: 17/932,775

(22) Filed: Sep. 16, 2022

Related U.S. Application Data
(60) Provisional application No. 63/245,662, filed on Sep. 17, 2021.

(51) Int. Cl. C12N 15/82

(52) U.S. Cl. CPC C12N 15/8265

(57) ABSTRACT

The present invention provides novel DNA molecules and constructs, including their nucleotide sequences, useful for modulating gene expression in plants and plant cells. The invention also provides DNA molecules and constructs, including their nucleotide sequences, useful for expressing proteins in plants to promote symbiotic infection. The invention also provides plants and plant cells transgenic plants, plant cells, plant parts, seeds, and commodity products comprising the DNA molecules operably linked to heterologous transcribable polynucleotides, along with methods of their use.

### CROSS REFERENCE TO RELATED APPLICATIONS

This application claims the benefit of U.S. Provisional Application No. 63/245,662, filed on Sep. 17, 2021, the entire content of which is hereby incorporated herein by reference.

### INCORPORATION OF SEQUENCE LISTING

The sequence listing that is contained in the file named “AGOE004US.xml”, which is 38.5 KB (as measured in Microsoft Windows®) and was created on Sep. 15, 2022, is filed herewith by electronic submission and is incorporated by reference herein.

### FIELD OF THE INVENTION

The invention relates to the field of plant molecular biology and plant genetic engineering, DNA molecules useful for modulating gene expression in plants, and proteins useful for improving agronomic performance.

### BACKGROUND

Many of the world's farmers also face pressure from nitrogen-deficient or phosphate-deficient soils which can result in low yield or plant death. Symbiotic bacteria can improve plant biomass under low-nitrogen conditions. Specifically, intracellular colonization of host cells by symbionts represents a mutualistic association that occurs between a host plant and soil-borne bacteria or fungi. However, intruding rhizobia bacteria cannot build morphological pre-infection cell surface attachment structures or form constricted structures that can physically support a developing infection against the plant cell turgor. Therefore, methods for forming and stabilizing membrane structures required for symbiotic infection in both legume and non-legume plants are needed to provide farmers with crop plants exhibiting improved agronomic performance under nitrogen-limited conditions.

### SUMMARY OF THE INVENTION

The invention provides DNA molecules and constructs, including their nucleotide sequences, useful for expressing proteins in plants to promote or enhance symbiotic infection. The proteins as disclosed herein can be used alone or in combination with other proteins in planta, thus providing alternatives means to form and stabilize membrane structures required for symbiotic infection. The present invention also provides novel DNA molecules and constructs, including their nucleotide sequences, useful for modulating gene expression in plants and plant cells. Furthermore, the invention also provides transgenic plants, plant cells, plant parts, seeds, and commodity products comprising the DNA molecules as described herein, along with methods of their use.

In one embodiment, disclosed in this application is a recombinant DNA molecule comprising a heterologous promoter operably linked to a polynucleotide segment encoding a lectin-domain containing protein or fragment thereof, wherein the lectin-domain containing protein comprises the amino acid sequence of SEQ ID NO:2 or SEQ ID NO:4-12; or the lectin-domain containing protein comprises an amino acid sequence having at least 85%, or 90%, or 95%, or 98% or 99%, or about 100% amino acid sequence identity to SEQ ID NO:2 or SEQ ID NO:4-12; or the polynucleotide segment hybridizes under stringent hybridization conditions to a polynucleotide having the nucleotide sequence of SEQ ID NO:1. The recombinant DNA molecule can comprise a sequence that functions to express the lectin-domain containing protein in a plant, and which when expressed in a plant cell produces a an increase in symbiotic infection of a bacteria or fungi.

In another embodiment of this application the recombinant DNA molecule is present within a bacterial or plant host cell. Contemplated bacterial host cells include at least the genus of Agrobacterium, Rhizobium, Bacillus, Brevibacillus, Escherichia, Pseudomonas, Klebsiella, Pantoea, and Erwinia. In certain embodiments, the Bacillus species is a Bacillus cereus or Bacillus thuringiensis, the Brevibacillus is a Brevibacillus laterosporus, or the Escherichia is a Escherichia coli. Contemplated plant host cells include a dicotyledonous plant cell and a monocotyledonous plant cell. Contemplated plant cells further include an alfalfa, banana, barley, bean, broccoli, cabbage, brassica, carrot, cassava, castor, cauliflower, celery, chickpea, Chinese cabbage, citrus, coconut, coffee, corn, clover, cotton (Gossypiurn sp.), a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat plant cell.

In another embodiment, the lectin-domain containing protein exhibits activity in the presence of bacteria or fungi, including rhizobia bacterium Mesorhizobium loti, Sinorhizobium meliloti, Sinorhizobium fredii, Rhizobium sp. IRBG74 and NGR234, Bradyrhizobium sp; and arbuscular mycorrhiza fungi Rhizophagus irregularis, Rhizophagus intraradices, Glomus mosseae, Funneliformis mosseae.

Also contemplated in this application are bacteria and plants and plant parts comprising a recombinant DNA molecule encoding the lectin-domain containing protein or fragment thereof. The recombinant molecule (e.g. construct) may comprise a heterologous promoter for expression in bacterial or plant cells of the operably linked polynucleotide segment encoding the lectin-domain containing protein. Both dicotyledonous plants and monocotyledonous plants are contemplated. In another embodiment, the plant is further selected from the group consisting of an alfalfa, banana, barley, bean, broccoli, cabbage, brassica (e.g. canola), carrot, cassava, castor, cauliflower, celery, chickpea, Chinese cabbage, citrus, coconut, coffee, corn, clover, cotton (i.e. Gossypiurn sp.), a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, corn (i.e. maize) such as sweet corn or field corn, sweet gum, sweet potato, switchgrass, tea, tobacco, tomato, triticale, turf grass, watermelon, and wheat. The plant parts may for instance include, without limitation, leaves, tubers, roots, stems, seeds, embryos, flowers, inflorescences, bolls, pollen, fruit, animal feed, and biomass. Processed plant parts, for instance wood, or oil, non-viable ground seeds or fractionated seeds, flour, or starch produced from the plant leaves, flowers, roots, seeds or tubers are also contemplated. Still further provided is a transgenic seed comprising the recombinant DNA molecules according to the invention.

In still yet another aspect, the invention provides a transgenic plant, or part thereof, further comprising a recombinant DNA molecule comprising a heterologous promoter operably linked to a polynucleotide segment encoding a protein or fragment thereof, wherein the protein comprises the amino acid sequence of SEQ ID NO:14 or SEQ ID NO:16-19; or the protein comprises an amino acid sequence having at least 85%, or 90%, or 95%, or 98% or 99%, or about 100% amino acid sequence identity to SEQ ID NO:14 or SEQ ID NO:16-19; or the polynucleotide segment hybridizes under stringent hybridization conditions to a polynucleotide having the nucleotide sequence of SEQ ID NO:13. The recombinant DNA molecule can comprise a sequence that functions to express the protein in a plant, and which when expressed in a plant cell stabilizes membrane curvature associated with symbiotic infection of a bacteria or fungi.

Commodity products comprising a detectable amount of the recombinant DNA molecules and disclosed proteins disclosed in this application are also contemplated. Such commodity products include commodity corn bagged by a grain handler, corn flakes, corn cakes, corn flour, corn meal, corn syrup, corn oil, corn silage, corn starch, corn cereal, and the like, and corresponding soybean, rice, wheat, sorghum, pigeon pea, peanut, fruit, melon, and vegetable commodity products including, where applicable, juices, concentrates, jams, jellies, marmalades, and other edible forms of such commodity products containing a detectable amount of such polynucleotides and or polypeptides of this application, whole or processed cotton seed, cotton oil, lint, seeds and plant parts processed for feed or food, fiber, paper, biomasses, and fuel products such as fuel derived from cotton oil or pellets derived from cotton gin waste, whole or processed soybean seed, soybean oil, soybean protein, soybean meal, soybean flour, soybean flakes, soybean bran, soybean milk, soybean cheese, soybean wine, animal feed comprising soybean, paper comprising soybean, cream comprising soybean, soybean biomass, and fuel products produced using soybean plants and soybean plant parts.

Also contemplated in this application is a method of producing seed comprising recombinant DNA molecules and the disclosed proteins. The method comprises planting at least one seed comprising the recombinant DNA molecules disclosed in this application; growing a plant from the seed; and harvesting seed from the plant, wherein the harvested seed comprises the referenced recombinant DNA molecules.

In another illustrative embodiment, a plant susceptible to symbiotic infection, is provided wherein the cells of said plant comprise the recombinant DNA molecules disclosed herein.

Also disclosed in this application are methods for increasing symbiotic infection in a plant, particularly a crop plant. The method comprises, in one embodiment, first expressing a lectin-domain containing protein or fragment thereof as set forth in any of SEQ ID NOs: 2 and 4-12 in a plant; or, alternatively, expressing a lectin-domain containing protein comprising an amino acid sequence having at least 85%, or 90%, or 95%, or 98% or 99%, or about 100% amino acid sequence identity to SEQ ID NO:2 and 4-12; and contacting said plant with an effective amount of one or more rhizobia bacterium, arbuscular mycorrhiza fungi, or a combination thereof. In certain embodiments, the method may further comprise, expressing a protein or fragment thereof as set forth in any of SEQ ID NO:14 and 16-19 in the plant; or alternatively, expressing a protein comprising an amino acid sequence having at least 85%, or 90%, or 95%, or 98% or 99%, or about 100% amino acid sequence identity to SEQ ID NO: 14 and 16-19.

In yet another aspect, the present invention provides a DNA molecule comprising a DNA sequence selected from the group consisting of: a) a sequence with at least 85 percent sequence identity to any of SEQ ID NO:3; b) a sequence comprising SEQ ID NO:3; and c) a fragment of SEQ ID NO:3, wherein the fragment has gene-regulatory activity; wherein said sequence is operably linked to a heterologous transcribable polynucleotide molecule. In specific embodiments, the DNA molecule comprises at least about 90 percent, at least about 95 percent, at least about 98 percent, or at least about 99 percent sequence identity to the DNA sequence of any of SEQ ID NOs: 3. In certain embodiments of the DNA molecule, the DNA sequence comprises a regulatory element. In some embodiments the regulatory element comprises a promoter. In certain embodiments of the DNA molecule, the sequence provides expression of the heterologous transcribable polynucleotide molecule in response to an external stimulus. In some embodiments the sequence provides expression of the heterologous transcribable polynucleotide molecule in a root hair cell.

Further provided by the invention is a transgenic plant, or part thereof, comprising a DNA molecule as provided herein, including a DNA sequence selected from the group consisting of: a) a sequence with at least 85 percent sequence identity to any of SEQ ID NO:3; b) a sequence comprising SEQ ID NO:3; and c) a fragment of SEQ ID NO:3, wherein the fragment has gene-regulatory activity; wherein the sequence is operably linked to a heterologous transcribable polynucleotide molecule. In specific embodiments, the transgenic plant may be a progeny plant of any generation that comprises the DNA molecule, relative to a starting transgenic plant comprising the DNA molecule. Still further provided is a transgenic seed comprising a DNA molecule according to the invention.

In still yet another aspect, the invention provides a method of expressing a transcribable polynucleotide molecule that comprises obtaining a transgenic plant according to the invention, such as a plant comprising a DNA molecule as described herein, and cultivating plant, wherein a transcribable polynucleotide in the DNA molecule is expressed.

Further aspects provided include plants or parts thereof comprising a recombinant DNA molecule as described herein encoding a lectin-domain containing protein or fragment thereof and further comprising a recombinant DNA molecule comprising a heterologous promoter operably linked to a polynucleotide segment encoding a protein or fragment thereof, wherein said protein comprises the amino acid sequence of SEQ ID NO: 21 or SEQ ID NO: 23; wherein said protein comprises an amino acid sequence having at least 85%, or 90%, or 95%, or 98% or 99%, or about 100% amino acid sequence identity to SEQ ID NO: 21 or SEQ ID NO: 23; or wherein said polynucleotide segment hybridizes under stringent hybridization conditions to a polynucleotide having the nucleotide sequence of SEQ ID NO: 20 or SEQ ID NO: 22. In further embodiments said plant further comprises a recombinant DNA molecule comprising a heterologous promoter operably linked to a polynucleotide segment encoding a protein or fragment thereof, wherein the protein comprises the amino acid sequence of SEQ ID NO:14 or SEQ ID NO:16-19; or the protein comprises an amino acid sequence having at least 85%, or 90%, or 95%, or 98% or 99%, or about 100% amino acid sequence identity to SEQ ID NO:14 or SEQ ID NO:16-19; or the polynucleotide segment hybridizes under stringent hybridization conditions to a polynucleotide having the nucleotide sequence of SEQ ID NO:13.

Yet further aspects include plants or parts thereof comprising a recombinant DNA molecule as described herein encoding a lectin-domain containing protein or fragment thereof and further comprising a recombinant DNA molecule comprising a heterologous promoter operably linked to a polynucleotide segment encoding a protein or fragment thereof, wherein said protein comprises the amino acid sequence of SEQ ID NO: 25; wherein said protein comprises an amino acid sequence having at least 85%, or 90%, or 95%, or 98% or 99%, or about 100% amino acid sequence identity to SEQ ID NO: 25; or wherein said polynucleotide segment hybridizes under stringent hybridization conditions to a polynucleotide having the nucleotide sequence of SEQ ID NO: 24. In further embodiments said plant further comprises a recombinant DNA molecule comprising a heterologous promoter operably linked to a polynucleotide segment encoding a protein or fragment thereof, wherein the protein comprises the amino acid sequence of SEQ ID NO:14 or SEQ ID NO:16-19; or the protein comprises an amino acid sequence having at least 85%, or 90%, or 95%, or 98% or 99%, or about 100% amino acid sequence identity to SEQ ID NO:14 or SEQ ID NO:16-19; or the polynucleotide segment hybridizes under stringent hybridization conditions to a polynucleotide having the nucleotide sequence of SEQ ID NO:13.

Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE FIGURES

FIG. 1 shows LDP1 localization to the infection chamber (IC) (Panel A) and the growing infection thread (IT) (Panel B).

FIG. 2 shows LDP1 promoter expression in deforming and curling root hairs. In particular, Panels A-C show that the ProLDP1:NLS-2×GFP reporter remains active in M. truncatula transgenic roots inoculated with S. meliloti.

FIG. 3 shows enhanced symbiotic performance of stable transgenic M. truncatula lines ectopically expressing LDP1. In particular, Panels A and B show a phenotypic analysis (nodule number per plant and infection threads per plant) of stable transgenic M. truncatula lines constitutively over-expressing (OE) LDP1. Plants were inoculated for 10 days with S. meliloti.

FIG. 4 shows roles of LDP1/LDP2 during primary infection. In particular, Panels A-C show a phenotypic analysis (nodules per plant, infection chambers per plant, and infection threads per plant) of stable transgenic M. truncatula CRISPR-CAS lines simultaneously targeting LDP1 and LDP2. Plants were inoculated for 10 days with S. meliloti.

FIG. 5 shows addition of labelled LDP1 to liposomes resulting in LDP1 clustering.

FIG. 6 shows LDP1-mediated membrane invaginations in protoplasts in the presence of rhizobia. In particular, Panels A and B show that N. benthamiana protoplasts ectopically expressing GFP-LDP1 exhibit membrane invaginations in the presence of S. meliloti.

FIG. 7 shows association of the LDP1 lectin domain with rhizobia in solution and polar association with a rhizobial exopolysaccharide matrix.

FIG. 8 shows that SYMREM1 functions in bacterial release from nodular infection threads. In particular, Panels A-C show illustrations indicating an infection thread (IT) with an infection droplet (ID; Panel A), rhizobial release (R) and symbiosome (S) formation (Panel B) and a symbiosome-filled cell (Panel C); CW: cell wall; N: nucleus; V: vacuole. Panels D-G show expression of a GFP-SYMREM1 fusion protein (green) that specifically labels IT membranes, accumulates at bacterial droplet structures, and symbiosome membranes (S. meliloti expressing a mCherry marker; red). White arrow (in F) indicates the bacterial release site. Panels H-M show Transmission Electron Microscopy showing normal rhizobial release into wild-type (WT, H) while bacteria are trapped inside the IT droplet in symrem1 mutants (Panels E-F). ID, infection droplet. Scanning Electron Microscopy showing normal rhizobial release into bacteroids (Panel K) while bacteria are trapped inside the infection droplet in symrem1 mutants (Panels L-M), Scale bars indicate 5 μm in (Panels D-G and J), 2 μm in (Panels H and I), 4 μm in K and 3 μm in (Panels L and M). A WT droplet structure is encircled by a magenta line and a bacterial release site is labelled by an arrow head (Panel H). Collapsed infection droplets in symrem1 mutants were encircled with a red line (Panels I, J, L and M).

FIG. 9 shows that SYMREM1 stabilizes confined membrane tubes during infection. In particular, spatially confined membrane tubes were found on wild-type IT release sites (arrow, Panel A) but not on ITs in symrem1 mutants (Panel B). Symbiosome membranes are loosely associated with released rhizobia (Panel C) or appear as empty spheres (Panel D) in symrem1 mutants. These patterns were confirmed by transmission electron microscopy for WT (Panel E) and symrem1 mutants (Panels F-H). In (Panels C-H), arrows indicate symbiosome membranes that are loosely associated with released rhizobia and arrow heads indicate empty membrane spheres. Membrane tubes were found on nodular ITs in the ipd3 mutant (Panel I), while ectopic expression of SYMREM1 greatly increased these tubular outgrowths in the ipd3 mutant (Panel J) but not on WT (Panel K). Arrows indicate the membrane tubes in (Panels I-K). Membranes were visualized by expressing the phosphatidylserine biosensor LactC2 (Panels A-D, I) or YFP-SYMREM1 (Panels J-K). Scale bars indicate 5 μm (Panels A-D; I-K) and 2 μm (Panels E-H). IT: infection thread; ID: infection droplet.

FIG. 10 shows that SYMREM1 stabilizes membrane tubulation and curvature in a cell wall-independent manner. N. benthamiana protoplasts ectopically expressing YFP-SYMREM1 develop multiple membrane tubes as shown by confocal laser-scanning (Panel A) and scanning electron (Panel B and close-up in Panel B′) microscopy within 2 hours after cell wall removal. These membrane tubes comprise a central actin strand (Panel C-Panel C″) with a tip localized formin protein SYFO1 (Panel D-Panel D″, white arrows). Wall-less control protoplasts expressing the membrane marker mCitrine-LTI6b re-inflated immediately after 30 minutes of micro-capillary-based indentation (Panel E), while those expressing SYMREM1 retained the induced membrane curvature (Panel F). Scales indicate 15 μm (Panel A) 10 μm (Panels B, C-D″), 5 μm (Panel B′) and 25 μm (Panels E-F).

FIG. 11 shows visualization of symbiotic membranes via phosphatidylserine (PS) reporter Lact-C2 labeling. (Panels A-C) Illustrations indicate an infection thread (IT) with an infection droplet (ID; Panel A), rhizobial release (R) and symbiosome (S) formation (Panel B) and a symbiosome-filled cell (Panel C); CW: cell wall; N: nucleus; V: vacuole. (Panels E-G) To visualize membrane structures, phosphatidylserine was labelled using a LactC2 biosensor in symbiotic membranes (S. meliloti expressing a mCherry marker; red). Scale bars indicate 5 μm in (Panels D-F).

FIG. 12 shows that SYMREM1 undergoes liquid-liquid phase separation in HEK-293T cells. In particular, HEK-293T cells were transfected with different truncated variants of SYMREM1. In darkness, all fusion proteins localized to the cytosol (Panels A-D) while blue light-induced oligomerization resulted in the reversible formation of phase-separated condensates by the full-length SYMREM1 (Panel A′) and the N-terminal IDR fused to the RemCA membrane anchor (Panel D′) but not when expressing the IDR alone (Panel B′) or a variant truncated by the RemCA peptide (Panel C′). The induced opto-condensates (here full-length SYMREM1) fused over time (magenta arrows, Panel E-Panel E″) under constant blue-light irradiation, a hallmark of LLPS. Scale bars indicate 3 μm.

FIG. 13 shows that SYMREM1 forms oligomeric alpha helical assemblies. (Panel a) Representative raw electron micrograph of purified, recombinant SYMREM1 stained with 2% uranyl acetate. Arrow heads indicate irregular protein bodies. Scale bar indicates 100 nm, experiments were performed twice with independently isolated recombinant SYMREM1 protein. (Panel b) 2D class averages derived from multivariate statistical analysis of all 389 particle images. Each class contains on average ten images. Class averages that show twisted features are marked with a white asterisk. Scale bar indicates 100 Å. (Panel c) Elution profile of recombinant His-SYMREM1. Molecular masses (in kDa) and positions of elution peaks for standard proteins are indicated with triangles on the top. Insert: SDS-PAGE after Coomassie staining of the purified His-SYMREM1 (labelled by an arrow); molecular masses of the pre-stained protein standards are indicated on the left in kDa. (Panel d) AlphaFold predictions for homodimers of SYMREM1 (left) and MtREM2.1 (right). One monomer is colored from blue at the N-terminus to red at the C-terminus, the other in white. The Extended helical regions of both remorins form highly similar, antiparallel dimers. (Panel e) Prediction of higher-order oligomers using AlphaFold2. The remorin homodimers form flexible sheets that can be extended into helical structures. (Panel f) Electrostatic surface potential maps for the two faces of the sheets formed by SYMREM1 (above) and MtREM2.1 (below), contoured from −5k_{B}T (red) to +5k_{B}T (blue). In both cases, the convex faces show a positive electrostatic potential, while that of the concave faces is negative. (Panel g) A predicated helical super-structure based on the oligomerization of SYMREM1 dimers.

FIG. 14 shows remorin-induced membrane tubulation in protoplasts. Membrane tubulation was not observed in protoplasts expressing the membrane marker mCitrine-LTI6b as a control (Panel A) but only in those expressing YFP-SYMREM1 (Panels B-D) or several other Arabidopsis remorins (Panels E-J), indicating this feature being evolutionary conserved within remorin family. SYMREM1 occasionally induced long and branched tubes (Panels C-D). (Panel K) The C-terminal coiled-coil region of SYMREM1 (SYMREM1^{CC}) is sufficient to induce membrane tubulation (Panel K) while membrane tubulation was abolished when expressing the N-terminal IDR (SYMREM1^{IDR}; Panel L), and a SYMREM1 variant truncated by the C-terminal membrane anchor (SYMREM1^{ΔRemCA}; Panel M). Scale bars indicate 10 μm.

FIG. 15 depicts a sequence alignment of the M. truncatula LDP1 protein Medtr5g031160.1 (SEQ ID NO: 2), the M. truncatula LDP2 protein Medtr5g031140.1 (SEQ ID NO: 4), and LDP1-related proteins Medtr5g031120.1 (SEQ ID NO: 5), Medtr5g031100.1 (SEQ ID NO: 6), Cicari_Ca 24344.1 (SEQ ID NO: 7), Glymax_Glyma.01G020700.1 (SEQ ID NO: 8), Cajcaj_Ccajan 12288 (SEQ ID NO: 9), Glymax_Glyma.09G201400.1 (SEQ ID NO: 10), Trisub_gene-TSUD_237380 gene=TSUD_237380 (SEQ ID NO: 11), and Trisub_gene-TSUD 237390 gene=TSUD 237390 (SEQ ID NO: 12). The proteins shown comprise a highly conserved lectin domain indicated by a bar.

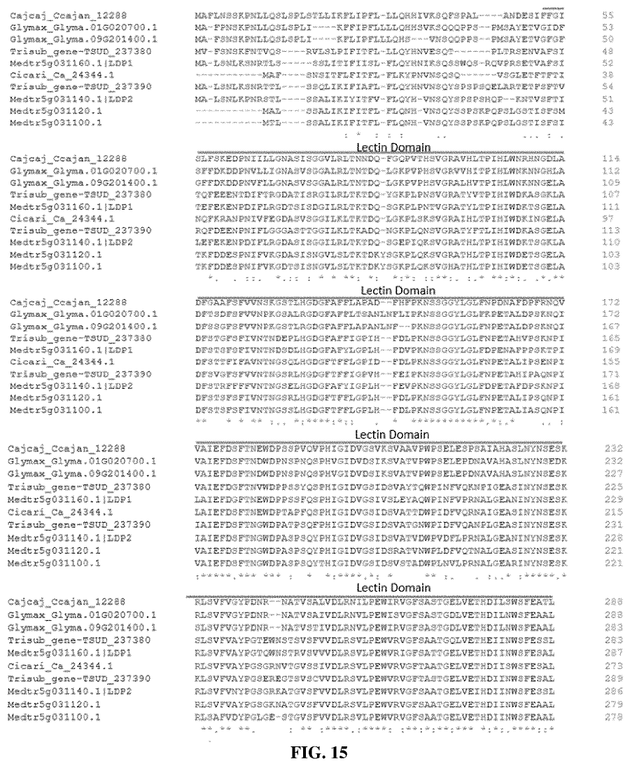

FIG. 16 is a sequence alignment of SYMREM1 proteins from Glycine max (labeled “Glycine”; SEQ ID NO: 19), Lotus japonicas (labeled “Lotus”; SEQ ID NO: 18), Cicer arietinum (labeled “Cicer”; SEQ ID NO: 16), Medicago truncatula (labeled “Medicago”; SEQ ID NO: 14), and Trifolium pratense (labeled “Trifolium”; SEQ ID NO: 17). The SYMREM1 proteins comprise a highly conserved C-terminal region (coiled-coil) (i.e. Conserved Remorin Domain) and a less conserved N-terminal segment, which is intrinsically disordered (ID).

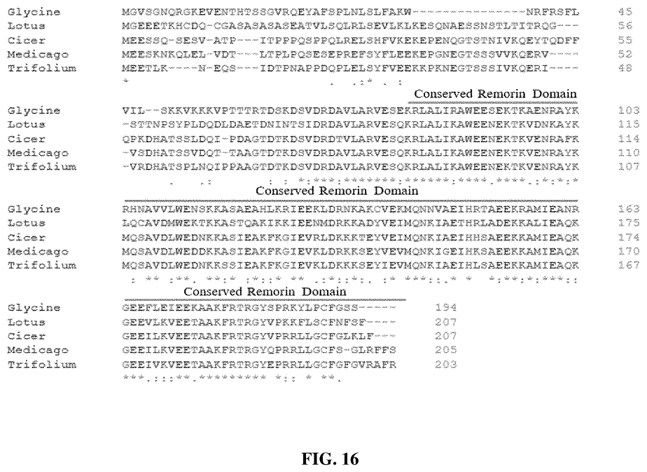

FIG. 17 shows activation of the Medicago LDP1 promoter in tomato hairy roots inoculated with arbuscular mycorrhiza (AM) fungi. Six-week old tomato hairy roots (Solanum lycopersicum cv. M82 WT*) were transformed with Agrobacterium rhizogenes strain Arqua1 carrying a MedtrLDPlpro::GUS//SolycACT2pro::NLS-2xmCherry construct. Tomato plants were transferred to vermiculite/sand pots containing AM fungi for two weeks. Roots were stained with X-gluc staining solution to observe GUS activity and AM structures were stained with WGA-Alexa Fluor 488. Scale bar 100 μm.

FIG. 18 shows nodule-like structure formation in tomato and tobacco hairy roots overexpressing NFP/LYK3 inoculated with rhizobia. Six-week-old tomato hairy roots (Solanum lycopersicum cv. Moneymaker) transformed with Arqua1 strain carrying the Medicago NFP/LYK3/proLDP1::GUS constructs. Tomato plants were transferred to vermiculite/sand pots and inoculated with S. meliloti (A to C), an S. meliloti and rhizobium mixture (D to E) or both (G to I) (OD ˜0.3) for 7 days. Transformed roots were selected (A, D and G). For GUS activity, transgenic roots were stained with X-gluc buffer (B, E and H) and 9-10 μm longitudinal sections of GUS-stained roots were further stained for 15 min in 0.1% Ruthenium Red (C, F and I). Scale bar 100 μm.

FIG. 19 Leaves were infiltrated with Agrobacteria and allowed to express either the AtREM3.2 construct or a Lti6b construct (as control). Isolation of protoplasts from these leaves resulted in a mixed population with high number of cells maintaining the jigsaw puzzle shape when expressing AtREM3.2. The lack of cell wall was determined by Calcofluor White staining.

FIG. 20 shows expression of an AtREM3.2/SYMREM1 chimera construct in tobacco leaf cells. Leaves were first infiltrated with Agrobacteria and allowed to express either the SYMREM1 C-terminal domain (SYMREM1^{C}) or the chimeric construct (AtREM3.2^{N}/SYMREM1^{C}). Isolation of protoplasts from these leaves resulted in a mixed population with high number of cells maintaining the jigsaw puzzle shape when expressing the chimeric construct (AtREM3.2^{N}/SYMREM1^{C}).

### BRIEF DESCRIPTION OF THE SEQUENCES

SEQ ID NO:1 is a nucleic acid sequence encoding the M. truncatula LDP1 protein Medtr5g031160.1.

SEQ ID NO: 2 is the amino acid sequence of the M. truncatula LDP1 protein Medtr5g031160.1, encoded by SEQ ID NO: 1.

SEQ ID NO: 3 is the nucleic acid sequence of the promoter sequence of the M. truncatula LDP1 protein Medtr5g031160.1.

SEQ ID NO: 4 is the amino acid sequence of the M. truncatula LDP2 protein Medtr5g031140.1.

SEQ ID NO: 5 is the amino acid sequence of the LDP1-related protein Medtr5g031120.1.

SEQ ID NO: 6 is the amino acid sequence of the LDP1-related protein Medtr5g031100.1.

SEQ ID NO: 7 is the amino acid sequence of the LDP1-related protein Cicari_Ca_24344.1.

SEQ ID NO: 8 is the amino acid sequence of the LDP1-related protein Glymax_Glyma.01G020700.1.

SEQ ID NO: 9 is the amino acid sequence of the LDP1-related protein Cajcaj_Ccajan_12288.

SEQ ID NO: 10 is the amino acid sequence of the LDP1-related protein Glymax_Glyma.09G201400.1.

SEQ ID NO: 11 is the amino acid sequence of the LDP1-related protein Trisub_gene-TSUD_237380 gene=TSUD_237380.

SEQ ID NO: 12 is the amino acid sequence of the LDP1-related protein Trisub_gene-TSUD_237390 gene=TSUD_237390.

SEQ ID NO: 13 is a nucleic acid sequence encoding the M. truncatula SYMREM1 protein.

SEQ ID NO: 14 is the amino acid sequence of the M. truncatula SYMREM1 protein, encoded by SEQ ID NO: 13.

SEQ ID NO: 15 is the nucleic acid sequence of the promoter sequence of the M. truncatula SYMREM1 protein.

SEQ ID NO: 16 is the amino acid sequence of the Cicer arietinum SYMREM1 protein.

SEQ ID NO: 17 is the amino acid sequence of the Trifolium pratense SYMREM1 protein.

SEQ ID NO: 18 is the amino acid sequence of the Lotus japonicus SYMREM1 protein.

SEQ ID NO: 19 is the amino acid sequence of the Glycine max SYMREM1 protein.

SEQ ID NO: 20 is the nucleic acid sequence encoding the Medicago Nod Factor Perception (NFP) receptor.

SEQ ID NO: 21 is the amino acid sequence of Medicago Nod Factor Perception (NFP) receptor.

SEQ ID NO: 22 is the nucleic acid sequence encoding Medicago Lysin Motif Receptor-Like Kinase3 (LYK3).

SEQ ID NO: 23 is the amino acid sequence of Medicago Lysin Motif Receptor-Like Kinase3 (LYK3).

SEQ ID NO: 24 is the nucleic acid sequence of the AtREM3.2 (At4g00670) gene.

SEQ ID NO: 25 is the amino acid sequence encoded by the AtREM3.2 (At4g00670) gene.

### DETAILED DESCRIPTION OF THE INVENTION

### LDP1 and SYMREM1

Improving crop yield from agriculturally significant plants has become increasingly important. In addition to the growing need for agricultural products to feed, clothe and provide energy for a growing human population, climate-related effects and pressures are predicted to reduce the amount of arable land available for farming. These factors have led to grim forecasts of food security, particularly in the absence of major improvements in plant biotechnology and agronomic practices. In light of these pressures, environmentally sustainable improvements in technology, agricultural techniques, and pest management are vital tools to expand crop production on the limited amount of arable land available for farming.

Many of the world's farmers also face pressure from nitrogen-deficient or phosphate-deficient soils which can result in low yield or plant death. Symbiotic bacteria can improve plant biomass under low-nitrogen conditions. Specifically, intracellular colonization of host cells by symbionts represents a mutualistic association that occurs between a host plant and soil-borne bacteria or fungi. For example, legumes are known to form a symbiotic relationship with nitrogen-fixing rhizobia, generally referred to as root nodule symbiosis (RNS). During RNS, host-derived perimicrobial membranes tightly align along the invading symbionts and separate them over the entire lifespan of the association from the host cell cytosol. However, intruding rhizobia cannot build pre-infection cell surface attachment structures such as hyphopodia as maintained by AM fungi (AMF), nor can they form physically constricted structures like invasive, tip-growing hyphae that can physically support a perimicrobial membrane tube against the plant cell turgor. Therefore, pre-infection membrane invaginations and constitutive infection thread (IT) stabilization during RNS is most likely host-driven process that enables symbiotic rhizobia to transcellularly progress through this preformed membrane tunnel by formative divisions. Proteins involved in the formation and stabilization membrane structures required for symbiotic infection are therefore provided herein, together with regulatory elements for advantageous spatial and temporal expression of such proteins.

Although the role of soluble lectin proteins during symbiotic infection has been previously investigated, more recent studies focused on the recognition of rhizobial NOD factors by receptor-like kinases (RLK), e.g. LysM, resulting in the initiation of kinase-dependent signaling cascades, ultimately leading to symbiotic infection.

The LDP1 and functionally redundant LDP2 proteins comprise extracellular lectin domains and transmembrane domains; however, LDP1 lacks an intracellular kinase domain and only comprises a short intracellular region. Due to the lack of an intracellular kinase domain, LDP1 and LDP2 would not be expected to be directly involved in the initiation of signaling cascades leading to symbiotic infection. However, surprisingly, as demonstrated herein, LDP1 can induce membrane invaginations in the presence of rhizobia by clustering LDP1 at the bilayer. Cells respond to such membrane tension and curvature by polarization, wherein they align their actin and later microtubule cytoskeleton towards the newly formed invagination. Temporally controlled transcription of secretion cargo (e.g. cell wall-degrading enzymes) can then be locally deposited at this site. The instant disclosure therefore provides recombinant DNA molecules comprising LDP1, LDP2, or other LDP1-related proteins, for example any of SEQ ID NO: 2 and 4-12, operably linked to a heterologous promoter. Plants heterologously expressing or overexpressing LDP1, LDP2, or other LDP1-related proteins, for example any of SEQ ID NOs: 2 and 4-12, which facilitate symbiosis by inducing membrane invagination in the presence of rhizobia are further provided.

As also described herein, the LDP1 promoter region tightly controls spatial and temporal expression of LDP1 during the symbiotic infection. Expression of proteins involved in symbiotic infection must be carefully orchestrated to allow infection to proceed and persist to produce plants with agronomically advantageous traits. The instant disclosure therefore further provides recombinant DNA molecules comprising the LDP1 promoter sequence or variants or fragments thereof, operably linked to a heterologous coding sequence. In certain examples, recombinant DNA molecules comprise SEQ ID NO: 3 or variants or fragments thereof operably linked to a heterologous transcribable DNA molecule. Such promoter sequences may be infection-specific, e.g. related to the perception of rhizobial exopolysaccharides, and may activate gene expression independent of Nod Factor receptors for spatial and temporal control of gene expression.

Additionally, symbiotic infection requires massive membrane rearrangements as the intracellular symbionts remain surrounded by a host derived perimicrobial membrane. Inwards plant plasma membrane curvatures must be structurally supported against the cellular turgor. The present disclosure demonstrates that SYMREM1 functions as a structural scaffold to stabilize membrane tubulations that are required for intracellular infection and symbiotic nitrogen fixation. Therefore, in some embodiments the present invention provides recombinant DNA molecules comprising SYMREM1 or variants or fragments thereof, for example any of SEQ ID NO: 14 and 16-19, or variants or fragments thereof, operably linked to a heterologous promoter. Plants heterologously expressing or overexpressing SYMREM1 proteins, for example any of SEQ ID NO: 14 and 16-19, or variants or fragments thereof, which stabilize membrane structure, are further provided.

The instant disclosure further provides recombinant DNA molecules as well as plants, plant cells, plant parts, or seeds comprising recombinant DNA molecules for expression or co-expression of Medicago Nod Factor Perception (NFP) receptor proteins or Medicago Lysin Motif Receptor-Like Kinase3 (LYK3). These plants, plant cells, plant parts, or seeds may comprise recombinant DNA molecules comprising SEQ ID NOs: 20 or 22, or fragments or variants thereof, or recombinant DNA molecules encoding SEQ ID NOs: 21 or 23, or fragments or variants thereof. Plants comprising DNA molecules encoding NFP receptor proteins and/or LYK3 proteins with or without SYMREM proteins are further contemplated. Plants of the present disclosure may additionally comprise DNA molecules encoding NFP receptor proteins and/or LYK3 proteins with or without SYMREM proteins and with or without LDP1 protein. For example, plants of the present disclosure comprise DNA molecules encoding SEQ ID NO: 21 and/or SEQ ID NO: 23 with or without any of SEQ ID NOs: 14 and 16-19 and with or without any of SEQ ID NO:s 2 and 4-12.

Also provided are recombinant DNA molecules as well as plants, plant cells, plant parts, or seeds comprising recombinant DNA molecules for expression of the AtREM3.2 (At4g00670) gene. These plants, plant cells, plant parts, or seeds may comprise recombinant DNA molecule comprising SEQ ID NO: 24, or fragments or variants thereof, or recombinant DNA molecules encoding SEQ ID NO: 25, or fragments or variants thereof. Plants comprising DNA molecules comprising AtREM3.2 with or without DNA molecules encoding SYMREM proteins are further contemplated. Plants of the present disclosure may additionally comprise DNA molecules comprising AtREM3.2 with or without DNA molecules encoding SYMREM and with or without DNA molecules encoding LDP1 protein. For example, plants of the present disclosure comprise DNA molecules encoding SEQ ID NO: 25 with or without any of SEQ ID NOs: 14 and 16-19 and with or without any of SEQ ID NOs: 2 and 4-12.

The instant disclosure further provides recombinant DNA molecules as well as plants, plant cells, plant parts, or seeds comprising recombinant DNA molecules for co-expression of LDP1 and SYMREM1 proteins. In certain embodiments, recombinant DNA molecules for expression of any of SEQ ID NO: 2 and 4-12, or variants or fragments thereof, together with recombinant DNA molecules for expression of any of SEQ ID NO: 14 and 16-19, are provided. When expressed together, from the same or separate DNA constructs, LDP1 and SYMREM1 proteins, variants, or fragments can promote symbiotic infection by effectively inducing membrane invagination in the presence of rhizobia and stabilizing membrane tubulations that are required for intracellular infection and symbiotic nitrogen fixation.

The instant disclosure further provides regulatory polynucleotide molecules capable of providing unique spatial and temporal expression of operably linked proteins. In certain embodiments, regulator polynucleotide molecules provided include SEQ ID NO: 3 or 15, or fragments or variants thereof. These polynucleotide molecules are, for instance, capable of affecting the expression of an operably linked transcribable polynucleotide molecule in plant tissues, and selectively regulating gene expression or activity of an encoded gene product in transgenic plants.

### Symbiotic Bacteria

The present invention provides DNA molecules encoding proteins that when expressed in a plant may promote symbiotic infection, or express a transcribable polynucleotide molecule in response to symbiotic infection. Rhizobia are bacteria found in soil that infect the roots of legumes and colonize root nodules which are involved in nitrogen utilization. As used herein, “rhizobia” refers to any diazotrophic bacteria that fix atmospheric nitrogen inside plants roots.

Symbiotic bacteria can be used with plants comprising the recombinant DNA molecules described herein to produce improved agronomic effects including improved plant growth or increased yield or biomass under reduced nitrogen conditions. Symbiotic bacteria useful with the disclosed plants include, but are not limited to, Mesorhizobium loti, Sinorhizobium meliloti, Sinorhizobium fredii, Rhizobium sp. IRBG74 and NGR234, Bradyrhizobium sp.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, a “recombinant DNA molecule” is a DNA molecule comprising a combination of DNA molecules that would not naturally occur together without human intervention. For instance, a recombinant DNA molecule may be a DNA molecule that is comprised of at least two DNA molecules heterologous with respect to each other, a DNA molecule that comprises a DNA sequence that deviates from DNA sequences that exist in nature, a DNA molecule that comprises a synthetic DNA sequence or a DNA molecule that has been incorporated into a host cell's DNA by genetic transformation or gene editing.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

A polynucleotide or polypeptide provided herein may further include two or molecules which are heterologous with respect to one another. As used herein, the term “heterologous” refers to the combination of two or more polynucleotide molecules or two or more polypeptide molecules when such a combination is not normally found in nature. For example, the two molecules may be derived from different species and/or the two molecules may be derived from different genes, e.g. different genes from the same species or the same genes from different species. In some examples, a promoter is heterologous with respect to an operably linked transcribable polynucleotide molecule if such a combination is not normally found in nature, i.e. that transcribable polynucleotide molecule is not naturally occurring operably linked in combination with that promoter molecule.

Any number of methods well known to those skilled in the art can be used to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragment thereof, can also be obtained by other techniques such as by directly synthesizing the fragment by chemical means, as is commonly practiced by using an automated oligonucleotide synthesizer.

As used herein, the term “percent sequence identity,” “percent identity,” or “% sequence identity” refers to the percentage of identical nucleotides or amino acids in a linear polynucleotide or polypeptide sequence of a reference (e.g. “query”) sequence (or its complementary strand) as compared to a test (e.g. “subject”) sequence (or its complementary strand) when the two sequences are optimally aligned. An optimal sequence alignment is created by manually aligning two sequences, e.g. a reference sequence and another sequence, to maximize the number of nucleotide matches in the sequence alignment with appropriate internal nucleotide insertions, deletions, or gaps. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar, Inc., 1228 S. Park St., Madison, Wis. 53715), and MUSCLE (version 3.6) (RC Edgar, Nucleic Acids Research (2004) 32(5):1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. As used herein, the term “sequence identity” refers to the extent to which two optimally aligned polynucleotide sequences or two optimally aligned polypeptide sequences are identical. As used herein, the term “reference sequence,” for example, may refer to a sequence provided as the polynucleotide sequences of any of SEQ ID NOs: 1, 3, 13, 15, 20, 22, and 24 or the polypeptide sequences of any of SEQ ID NOs: 2, 4-12, 14, 16-19, 21, 23, and 25.

Thus, one embodiment of the invention is a recombinant DNA molecule comprising a sequence that when optimally aligned to a reference sequence, provided herein as the polynucleotide sequences of SEQ ID NOs: 1, 3, 13, 15, 20, 22, and 24 has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having the activity of the reference sequence, for example the activity of any of SEQ ID NOs 1, 3, 13, 15, 20, 22, and 24.

Similarly, another embodiment of the invention is a polypeptide molecule comprising a sequence that when optimally aligned to a reference sequence, provided herein as the polypeptide sequences of SEQ ID NOs: 2, 4-12, 14, 16-19, 21, 23, and 25 has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having the activity of the reference sequence, for example the activity of any of SEQ ID NOs: 2, 4-12, 14, 16-19, 21, 23, and 25.

Also provided are fragments of polynucleotide sequences provided herein, for example fragments of a polynucleotide sequence selected from SEQ ID NOs: 1, 3, 13, 15, 20, 22, and 24. In specific embodiments, fragments of a polynucleotide sequences are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule of any of SEQ ID NOs: 1, 3, 13, 15, 20, 22, and 24. Methods for producing such fragments from a starting molecule are well known in the art. Fragments of a polynucleotide sequence provided herein may comprise the activity of the base sequence.

Disclosed sequences may hybridize specifically to a target DNA sequence under stringent hybridization conditions. In certain embodiments, polynucleotides disclosed herein may hybridize under stringent hybridization conditions to a polynucleotide having the nucleotide sequence of SEQ ID NO: 1 or 13. Stringent hybridization conditions are known in the art and described in, for example, M R Green and J Sambrook, Molecular cloning: a laboratory manual, 4^{th} Edition, Cold Spring Harbor Laboratory Press, Cold Spring Harbor, N.Y. (2012). As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Recombinant polynucleotide sequences encoding fragments of polypeptide sequences provided herein are further envisioned, including polynucleotide sequences encoding fragments of a polypeptide sequence selected from SEQ ID NOs: 2, 4-12, 14, 16-19, 21, 23, and 25. In specific embodiments, fragments of a polypeptide are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous amino acids, or longer, of a polypeptide molecule selected from SEQ ID NOs: 2, 4-12, 14, 16-19, 21, 23, and 25. Methods for producing such fragments from a starting molecule are well known in the art. Fragments of a polynucleotide sequence provided herein may maintain the activity of the base sequence.

### Transcribable Polynucleotide Molecules

Recombinant DNA molecules provided herein include transcribable polynucleotide molecules or sequences encoding useful polypeptide sequences. In certain examples, transcribable polynucleotide molecules include sequences encoding LDP1, LDP2, or LDP1-related polypeptides. Transcribable polynucleotides provided herein include SEQ ID NO: 1, or polynucleotide sequences encoding any of SEQ ID NOs: 2 and 4-12, or fragments or variants thereof. In other examples, transcribable polynucleotide molecules include sequences encoding SYMREM1 or SYMREM1-related proteins. Transcribable polynucleotides provided herein include SEQ ID NO: 13, or polynucleotide sequences encoding any of SEQ ID NOs: 14 and 16-19, or fragments or variants thereof. Transcribable polynucleotides may also include polynucleotide molecules encoding Nod Factor Perception (NFP) receptor-related polypeptides such as SEQ ID NO: 21, or fragments or variants thereof, or polynucleotide molecules encoding Lysin Motif Receptor-Like Kinase3 (LYK3)-related polypeptides such as SEQ ID NO: 23, or fragments or variants thereof. Transcribable polynucleotides may also include polynucleotide molecules comprising AtREM3.2 (At4g00670)-related genes such as SEQ ID NO: 24, or fragments or variants thereof.

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

With respect to polypeptide sequences, the term “variant” as used herein refers to a second polypeptide sequence that is in composition similar, but not identical to, a first polypeptide sequence and yet the second polypeptide sequence still maintains the general functionality, i.e. same or similar activity, of the first polypeptide sequence. A variant may be a shorter or truncated version of the first polypeptide sequence and/or an altered version of the sequence of the first polypeptide sequence, such as one with different amino acid deletions, substitutions, and/or insertions. Variants having a percent identity to a sequence disclosed herein may have the same activity as the base sequence. For example, the transcribable polynucleotide molecule can encode a protein or variant of a protein or fragment of a protein that is functionally defined to maintain activity in transgenic host cells including plant cells, plant parts, explants and whole plants.

Similarly, with respect to polynucleotide sequences, the term “variant” as used herein refers to a second polynucleotide sequence that is in composition similar, but not identical to, a first polynucleotide sequence and yet the second polynucleotide sequence still maintains the general functionality, i.e. same or similar activity, of the first polynucleotide sequence. A variant may be a shorter or truncated version of the first polynucleotide sequence and/or an altered version of the sequence of the first polynucleotide sequence, such as one with different nucleotide deletions, substitutions, and/or insertions. Variants having a percent identity to a sequence disclosed herein may have the same activity as the base sequence. For example, variant polynucleotides may encode the same or a similar protein sequence or have the same or similar gene regulatory activity as the base sequence.

As used herein, “modulation” of expression refers to the process of effecting either overexpression or suppression of a polynucleotide or a protein.

As used here, the term “overexpression” as used herein refers to an increased expression level of a polynucleotide or a protein in a plant, plant cell or plant tissue, compared to expression in a wild-type plant, cell or tissue, at any developmental or temporal stage for the gene. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

Overexpression can be achieved using numerous approaches. In one embodiment, overexpression can be achieved by placing the DNA sequence encoding one or more polynucleotides or polypeptides under the control of a promoter, examples of which include but are not limited to endogenous promoters, homologous promoters, heterologous promoters, inducible promoters, development specific promoters, and tissue specific promoters. In one exemplary embodiment, the promoter is a constitutive promoter, for example, the cauliflower mosaic virus 35S promoter and other constitutive promoters known in the art. Thus, depending on the promoter used, overexpression can occur throughout a plant, in specific tissues of the plant, in specific stages of development of the plant, or in the presence or absence of different inducing or inducible agents, such as hormones or environmental signals.

In certain embodiments, the expression or overexpression of a transcribable polynucleotide molecule encoding a protein as disclosed herein can affect an enhanced trait or altered phenotype directly or indirectly. In the latter case it may do so, for example, by promoting symbiotic infection process. In an exemplary embodiment, the protein produced from the transcribable polynucleotide molecule can stabilize membrane invaginations, e.g., to enhance intracellular infection and subsequently increase symbiotic nitrogen fixation.

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, product, nutritional profile, disease or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. The product of a gene of agronomic interest may act within the plant in order to cause an effect upon the plant physiology or metabolism.

In certain examples provided herein, a “gene of agronomic interest” also refers to transcribable polynucleotide molecules involved in symbiotic infection. For example, such genes of agronomic interest may include Nodule Pectate Lyase (NPL), Symbiotic Remorin 1 (SYMREM1), Rhizobium-directed polar growth (RPG), Interacting Protein of DMI3 (IPD3), and CYCLOPS.

In one embodiment of the invention, a promoter is incorporated into a construct such that the promoter is operably linked to a transcribable polynucleotide molecule that encodes an LDP1 protein, an LDP2 protein, or an LDP1-related protein, including any of SEQ ID NO: 2 and 4-12 or fragments or variants thereof. The expression of the transcribable polynucleotide molecule is desirable in order to confer an agronomically beneficial trait, including but not limited to improved capacity for symbiotic infection. An agronomically beneficial trait may also be, for example, modified yield, improved plant growth and development, improved biomass, increased resistance to environmental stress (e.g. nitrogen limited conditions), improved nitrogen fixation, improved fungal disease resistance, improved virus resistance, improved nematode resistance, improved bacterial disease resistance, improved starch production, modified oil production, modified fatty acid content, improved protein production, improved fruit ripening, enhanced animal and human nutrition, improved seed production, improved fiber production, and improved biofuel production.

Transcribable polynucleotide molecules may also be marker useful in detecting transformed plant cells, plant tissue, plant parts, or plants described herein. As used herein the term “marker” refers to any transcribable polynucleotide molecule whose expression, or lack thereof, can be screened for or scored in some way. Marker genes for use in the practice of the present invention include, but are not limited to transcribable polynucleotide molecules encoding β-glucuronidase (GUS described in U.S. Pat. No. 5,599,670), red fluorescent protein (e.g. mCherry), green fluorescent protein and variants and derivatives thereof (GFP described in U.S. Pat. Nos. 5,491,084 and 6,146,826), proteins that confer antibiotic resistance, or proteins that confer herbicide tolerance. Useful antibiotic resistance markers, including those encoding proteins conferring resistance to Basta (bar), kanamycin (nptII), hygromycin B (aph IV), streptomycin or spectinomycin (aad, spec/strep) and gentamycin (aac3 and aacC4) are known in the art. Herbicides for which transgenic plant tolerance has been demonstrated and the method of the present invention can be applied, include, but are not limited to: amino-methyl-phosphonic acid, glyphosate, glufosinate, sulfonylureas, imidazolinones, bromoxynil, dalapon, dicamba, cyclohexanedione, protoporphyrinogen oxidase inhibitors, and isoxasflutole herbicides.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g. by ELISA), small active enzymes which are detectable in extracellular solution (e.g, alpha-amylase, beta-lactamase, phosphinothricin transferase), or proteins which are inserted or trapped in the cell wall (such as proteins which include a leader sequence such as that found in the expression unit of extension or tobacco pathogenesis related proteins also known as tobacco PR-S). Other possible selectable marker genes will be apparent to those of skill in the art and are encompassed by the present invention.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. The term includes an expression cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention. For example, Agrobacterium rhizogenes ARqua1.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N.

Irwin, Cold Spring Harbor Laboratory Press. Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, Fla. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

Various regulatory elements may be included in a construct including any of those provided herein such as SEQ ID NO: 3 or 15, or variants or fragments thereof. Any such regulatory elements may be provided in combination with other regulatory elements. Such combinations can be designed or modified to produce desirable regulatory features. In one embodiment, constructs of the present invention comprise at least one regulatory element operably linked to a transcribable polynucleotide molecule operably linked to a 3′ UTR.

Constructs of the present invention may include any promoter or fragment or variant thereof provided herein, such as SEQ ID NO: 3 or 15, or known in the art. For example, a promoter of the present invention may be operably linked to a transcribable polynucleotide sequence, such as a sequence encoding one or more polypeptides selected from SEQ ID NOs: 2, 4-12, 14, and 16-19, or variants or fragments thereof. Alternatively, a heterologous promoter such as the Cauliflower Mosaic Virus 35S transcript promoter (see, U.S. Pat. No. 5,352,605) may be operably linked to a polypeptide sequence as disclosed herein.

A construct provided herein may further comprise additional elements useful in regulating or modulating expression of a transcribable polynucleotide, including leader, enhancer, intron, and 3′ UTR sequences. A construct provided herein may further comprise one or more marker sequences for identification of the construct in plant cells, plant tissue, or plants.

### Regulatory Elements

A regulatory element is a DNA molecule having gene regulatory activity, i.e. one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a regulatory element may be comprised of expression elements, such as enhancers, promoters, and introns, operably linked. A regulatory element may also be comprised of leaders and 3′ untranslated regions (3′ UTRs). Regulatory elements, capable of providing a unique spatial and temporal expression profile to an operably linked heterologous transcribable polynucleotide molecule are therefore useful for modifying plant phenotypes through the methods of genetic engineering. Regulatory elements include SEQ ID NOs: 3 and 15 provided herein, or variants and fragments thereof.

Regulatory elements may be characterized by their expression pattern effects (qualitatively and/or quantitatively), e.g. positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory element for modulating the expression of an operably linked transcribable polynucleotide molecule.

As used herein, the term “expression pattern” or “expression profile” is any pattern of translation of a transcribed RNA molecule into a protein molecule. Protein expression may be characterized by its temporal, spatial, developmental, or morphological qualities as well as by quantitative or qualitative indications.

As used herein, the term “promoter” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules. Promoters useful in practicing the present invention include SEQ ID NOs: 3 and 15, or variants or fragments thereof.

In specific embodiments of the invention, such molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for initiation of transcription. In accordance with the invention a promoter or promoter fragment may be analyzed for the presence of known promoter elements, i.e. DNA sequence characteristics, such as a TATA-box and other known transcription factor binding site motifs. Identification of such known promoter elements may be used by one of skill in the art to design variants of the promoter having a similar expression pattern to the original promoter.

In one embodiment variants of the disclosed promoter sequences are provided. For example, a recombinant DNA molecule comprising a sequence that when optimally aligned to a reference sequence, provided herein as the polynucleotide sequences of SEQ ID NO: 3 or 15, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having the activity of the reference sequence, for example the activity of any of SEQ ID NOs: 3 or 15.

Also provided are fragments of regulatory sequences provided herein, for example fragments of a polynucleotide sequence selected from SEQ ID NOs: 3 and 15. In specific embodiments, fragments of a polynucleotide sequences are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule of any of SEQ ID NOs: 3 and 15. Methods for producing such fragments from a starting molecule are well known in the art. Fragments of a polynucleotide sequence provided herein may comprise the activity of the base sequence.

As used herein, the term “chimeric” refers to a single DNA molecule produced by fusing a first DNA molecule to a second DNA molecule, where neither the first nor second DNA molecule would normally be found in that configuration, i.e. fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present disclosure.

### Plants Comprising DNA Molecules

Constructs, expression cassettes, and vectors comprising DNA molecules as disclosed herein can be constructed and introduced into a plant cell in accordance with transformation methods and techniques known in the art. For example, Agrobacterium-mediated transformation is described in U.S. Patent Application Publications 2009/0138985A1 (soybean), 2008/0280361A1 (soybean), 2009/0142837A1 (corn), 2008/0282432 (cotton), 2008/0256667 (cotton), 2003/0110531 (wheat), 2001/0042257 A1 (sugar beet), U.S. Pat. No. 5,750,871 (canola), 7,026,528 (wheat), and 6,365,807 (rice), and in Arencibia et al. (1998) Transgenic Res. 7:213-222 (sugarcane) all of which are incorporated herein by reference in their entirety. Transformed cells can be regenerated into transformed plants that express the polypeptides disclosed herein and demonstrate activity through bioassays as described herein as well as those known in the art. Plants can be derived from the plant cells by regeneration, seed, pollen, or meristem transformation techniques. Methods for transforming plants are known in the art.

The term “plant cell” or “plant” can include but is not limited to a dicotyledonous or monocotyledonous plant. In certain embodiments, plants provided herein are legumes, including, but not limited to, beans, soybeans, peas, chickpeas, peanuts, lentils, lupins, mesquite, carob, tamarind, alfalfa, and clover. Plants provided herein may also be non-legume plants.

The term “plant cell” or “plant” can also include but is not limited to an alfalfa, banana, barley, bean, broccoli, cabbage, brassica (e.g canola), carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, legumes, non-legumes, lettuce, Loblolly pine, millets, melons, nut, oat, olive, onion, ornamental, palm, pasture grass, pea, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, corn (i.e. maize, such as sweet corn or field corn, sweet gum, sweet potato, switchgrass, tea, tobacco, tomato, triticale, turf grass, watermelon, and wheat plant cell or plant.

In certain embodiments, transgenic plants and transgenic plant parts regenerated from a transgenic plant cell are provided. In certain embodiments, the transgenic plants can be obtained from a transgenic seed, by cutting, snapping, grinding or otherwise disassociating the part from the plant. In certain embodiments, the plant part can be a seed, a boll, a leaf, a flower, a stem, a root, or any portion thereof, or a non-regenerable portion of a transgenic plant part. As used in this context, a “non-regenerable” portion of a transgenic plant part is a portion that cannot be induced to form a whole plant or that cannot be induced to form a whole plant that is capable of sexual and/or asexual reproduction. In certain embodiments, a non-regenerable portion of a plant part is a portion of a transgenic seed, boll, leaf, flower, stem, or root.

The term “transformation” refers to the introduction of a DNA molecule into a recipient host. As used herein, the term “host” refers to bacteria, fungi, or plants, including any cells, tissues, organs, or progeny of the bacteria, fungi, or plants. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is inherited by subsequent progeny. A “transgenic” or “transformed” cell or organism may also include progeny of the cell or organism and progeny produced from a breeding program employing such a transgenic organism as a parent in a cross and exhibiting an altered phenotype resulting from the presence of a foreign DNA molecule. The introduced DNA molecule may also be transiently introduced into the recipient cell such that the introduced DNA molecule is not inherited by subsequent progeny. The term “transgenic” refers to a bacterium, fungus, or plant containing one or more heterologous DNA molecules.

There are many methods well known to those of skill in the art for introducing DNA molecules into plant cells. The process generally comprises the steps of selecting a suitable host cell, transforming the host cell with a vector, and obtaining the transformed host cell. Methods and materials for transforming plant cells by introducing a plant construct into a plant genome in the practice of this invention can include any of the well-known and demonstrated methods. Suitable methods include, but are not limited to, bacterial infection (e.g., Agrobacterium), binary BAC vectors, direct delivery of DNA (e.g., by PEG-mediated transformation, desiccation/inhibition-mediated DNA uptake, electroporation, agitation with silicon carbide fibers, and acceleration of DNA coated particles), gene editing (e.g., CRISPR-Cas systems), among others.

Host cells may be any cell or organism, such as a plant cell, algal cell, algae, fungal cell, fungi, bacterial cell, or insect cell. In specific embodiments, the host cells and transformed cells may include cells from crop plants.

A transgenic plant subsequently may be regenerated from a transgenic plant cell of the invention. Using conventional breeding techniques or self-pollination, seed may be produced from this transgenic plant. Such seed, and the resulting progeny plant grown from such seed, will contain the recombinant DNA molecule of the invention, and therefore will be transgenic.

Transgenic plants of the invention can be self-pollinated to provide seed for homozygous transgenic plants of the invention (homozygous for the recombinant DNA molecule) or crossed with non-transgenic plants or different transgenic plants to provide seed for heterozygous transgenic plants of the invention (heterozygous for the recombinant DNA molecule). Both such homozygous and heterozygous transgenic plants are referred to herein as “progeny plants.” Progeny plants are transgenic plants descended from the original transgenic plant and containing the recombinant DNA molecule of the invention. Seeds produced using a transgenic plant of the invention can be harvested and used to grow generations of transgenic plants, i.e., progeny plants of the invention, comprising the construct of this invention and expressing a gene of agronomic interest. Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

The transformed plants may be analyzed for the presence of the gene or genes of interest and the expression level and/or profile conferred by the regulatory elements of the invention. Those of skill in the art are aware of the numerous methods available for the analysis of transformed plants. For example, methods for plant analysis include, but are not limited to, Southern blots or northern blots, PCR-based approaches, biochemical analyses, phenotypic screening methods, field evaluations, and immunodiagnostic assays. The expression of a transcribable DNA molecule can be measured using TaqMan® (Applied Biosystems, Foster City, Calif.) reagents and methods as described by the manufacturer and PCR cycle times determined using the TaqMan® Testing Matrix. Alternatively, other methods and reagents for measuring expression of a transcribable DNA molecule are well known in the art. For example, the Invader® (Third Wave Technologies, Madison, Wis.) or SYBR Green (Thermo Fisher, A46012) reagents and methods as described by the manufacturer can be used to evaluate transgene expression.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. Progeny from these plants are evaluated, among other things, for gene expression. The gene expression may be detected by several common methods such as western blotting, northern blotting, immuno-precipitation, and ELISA.

As an alternative to traditional transformation methods, a DNA molecule, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

### Commodity Products

The present invention provides a commodity product comprising DNA molecules according to the invention. As used herein, a “commodity product” refers to any composition or product which is comprised of material derived from a plant, seed, plant cell or plant part comprising a DNA molecule of the invention. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the invention can thus be used to manufacture any commodity product typically acquired from plants or parts thereof.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: LDP1 Localizes to the Infection Chamber (IC) and the Growing Infection Thread (IT)

In order to investigate a role for LDP1 in symbiotic infection, LDP1 localization in stably transformed M. truncatula lines and transformed root hairs was visualized.

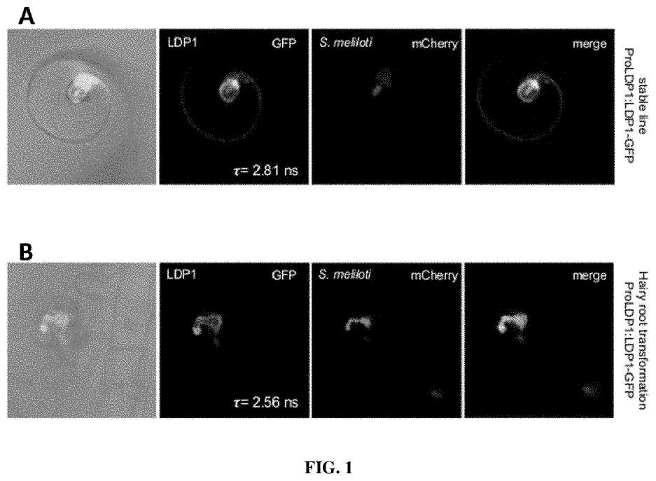

M. truncatula plant lines stably transformed with a construct comprising LDP1 and a GFP reporter gene (pL2-proNOS-Bar-tNOS-ins-proLDP1-LDP1-eGFP-t35 s) were created. 7 days old LDP1 stable transgenic seedlings were then inoculated with S. meliloti in open pots filled with a sand/vermiculite mixture as growth substrate for another 10 days. As shown in FIG. 1A, LDP1 localized to the infection chamber in the presence of S. meliloti.

Selected positive plants from hairy root transformation (proLjUBI-nls-2xmCherry-t35s-ins-proLDP1-LDP1-eGFP-t35s) were transferred to growth in open pots. These plants were inoculated around 4 days after transfer and images were taken 10 days post inoculation. As shown in FIG. 1B, LDP1 localized to the growing IT in the presence of S. meliloti.

These results indicate that LDP1 localizes to the IC and growing IT, the initial entry point for symbiotic infection.

### Example 2: The LDP1 Promoter Remains Active in Curling Root Hairs

In order to further assess the spatial and temporal expression of LDP1, a construct comprising the LDP1 promoter, the LDP1 coding sequence, and a GFP reporter sequence was expressed in M. truncatula transgenic roots inoculated with S. meliloti.

For the LDP1 promoter sequence, a 2000 bp fragment upstream of the start codon of LDP1 was amplified from M. truncatula genomic DNA. The coding sequence for LDP1 was amplified from M. truncatula inoculated root cDNA. For stable lines, all the sequences were synthesized. The reporter construct used in this example, and all the LII constructs (proLDP1-NLS-2×CFP, proLjUBI-GFP-LDP1, proLDP1-LDP1-GFP) and LIII constructs (proLDP1-NLS-2×CFP//proLjUBI-NLS-2xmCherry, proLDP1-LDP1-GFP//proLjUBI-NLS-2xmCherry) used were based on the GoldenGate cloning system.

To prepare samples for hairy root transformation, seeds of M. truncatula were surface sterilized by pure sulfuric acid (H₂S₅O₄) for 10-15 minutes, followed by 4 to 6 times washing with sterile Tap water. Then treated with bleaching solution (12% NaOCl, 0.1% SDS) for no longer than 1 min and washed with sterile water for 4 to 6 times. After sterilization, seeds were kept in water for around one hour then transferred to 1% water agar plates and stratified at 4° C. for 2-3 days in darkness. Germination was allowed for up to 24h at 24° C. in darkness. After seed germination, the seed coat was removed under water by a soft tweezers and the seedlings were transferred to Fahraeus medium plates supplemented with 0.5 mM NH₄NO₃ or cut off the root meristem by a scalpel for M. truncatula hairy root transformation (Medicago handbook, https://www.noble.org/medicago-handbook).

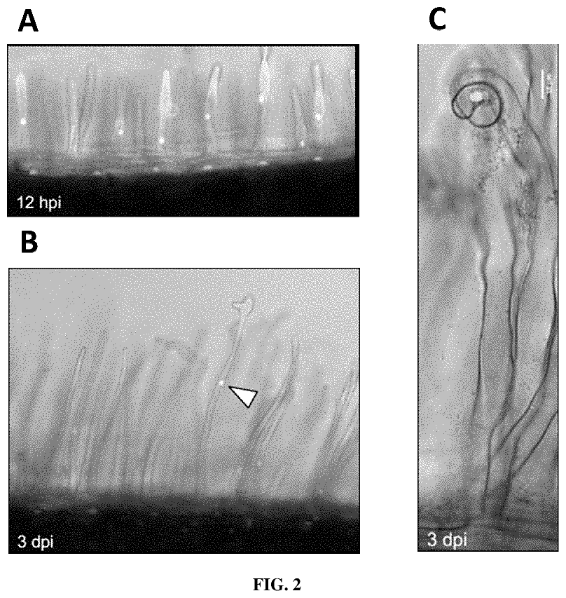

FIG. 2 shows expression of LDP1 M. truncatula transgenic roots inoculated with S. meliloti at 12 hours post inoculation and 3 days post-inoculation, demonstrating that the LDP1 promoter is active in curling root hairs. Using a proLDP1:NLS-2×GFP (proLjUBI-nls-2xmCherry//proLDP1-LDP1-eGFP-t35s) reporter construct revealed that LDP1 is globally induced in root hairs at 12 hpi (FIG. 2A) while the signal is later (3 dpi) exclusively maintained in curled and infected root cells (FIG. 2B and FIG. 2C). That is, the LDP1 promoter is inactive in the absence of rhizobia and is activated (at least 12 hours) after inoculation of the roots with rhizobia. However, when root hairs are initially infected, promoter activity is restricted to very few cells. This allows and unambiguous identification of infection-competent cells prior to root hair curling and throughout the infection process. Thus, the LDP1 promoter can provide spatial and temporal control gene expression.

### Example 3: Enhanced Symbiotic Performance of Stable Transgenic Lines Ectopically Expressing LDP1

To assess the effects of constitutively overexpressing LDP1 on symbiotic performance, stably transformed M. truncatula lines constitutively overexpressing LDP1 were inoculated for 10 days with S. meliloti. Phenotypic and molecular assays were performed.

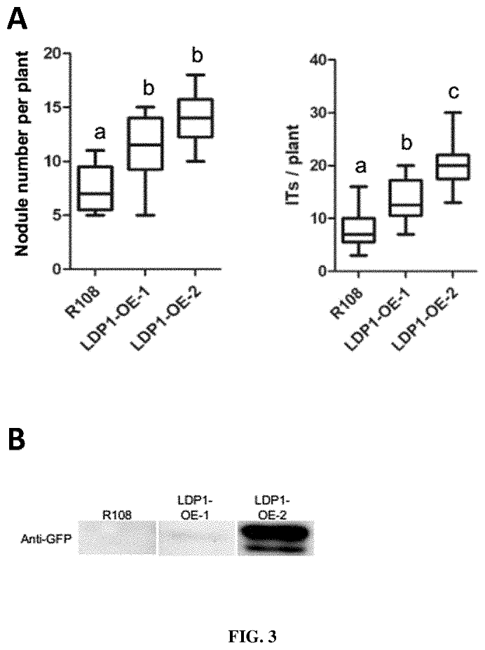

As shown in FIG. 3A, over-expression of LDP1 (proNOS-Bar-tNOS//proLiUBI-eGFP-LDP1-t35s) resulted in increased numbers of nodules (FIG. 3A) and infection threads at 10 dpi with S. meliloti. Protein levels of LDP1 in stable transgenic lines were also checked by Western blot analysis (FIG. 3B). The number of nodules per plant as well as the number of infection threads per plant were significantly increased in the transgenic M. truncatula lines over expressing LDP1 compared to the control line (R108).

### Example 4: LDP1/LDP2 Control Primary Infections

To further investigate the role of LDP1 and LDP2 in primary infection, stable transgenic M. truncatula CRISPR-CAS lines simultaneously targeting LDP1 and LDP2 were generated.

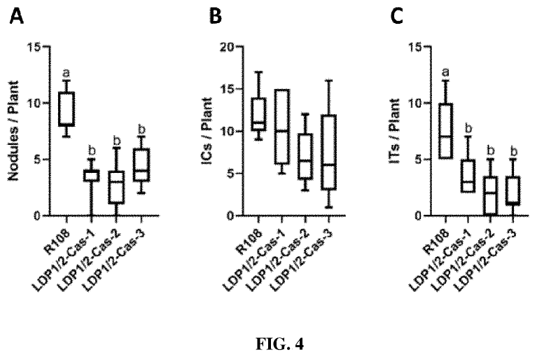

As shown in FIG. 4, phenotypic analysis revealed significant differences in transgenic CRISPR-CAS LDP1/LDP2 double mutant plants compared to the control line (R108) at 10 dpi with S. meliloti (grown in open pots).

Specifically, the numbers of nodules (FIG. 4A) and infection threads were significantly reduced (FIG. 4C), while the numbers of infection chambers (FIG. 4B) were not changed.

These results demonstrate that LDP1 and LDP2 play a role in promoting root nodule development as well as the formation of infection threads in plants treated with S. meliloti.

### Example 5: LDP1 Induces Negative Membrane Curvature In Vitro

In order to assess the effect of LDP1 expression on membrane curvature in vitro, labelled LDP1 proteins were added to liposome solution alone; and in the presence of rhizobia.

His fusion proteins were expressed and purified from E. coli BL21 cells using common laboratory techniques. Briefly, isopropyl β-D-1-thiogalactopyranoside (IPTG) was added to a final concentration of 1 mM in the bacterial liquid culture when its growth reach OD600=0.6, then incubated overnight at 16° C. Cells were harvested and resuspended in 100 ml Lysis buffer. After disruption with Constant Cell Disrupter (Constant Systems Limited), the cell debris was removed by centrifugation at 30,000 g for 30 min. The cleared cell lysate was loaded onto IMAC column (5 ml HisTrap_FF) pre-equilibrated and washed with 10 CV of loading buffer. The protein was eluted with a linear gradient from 20 to 450 mM (0-75%) imidazole in 15 CV. The eluted fractions were collected and concentrated by spin filtration to 5 ml. The concentrated protein was centrifuged for 10 min at 10,000 g before being loaded onto gel-filtration column (HiLoad Superdex 200 16/60) due to the precipitation of the protein.

Prepared liposomes contained DOPC (65%), cholesterol (30%), and DGS-NTA(Ni) lipids (5%) (DOPC-TR was used as a membrane marker). The purified lectin domain from LDP1 was labeled with an Atto488 NHS ester. The labeled protein was then incubated with liposomes (comprising NTA(Ni) associated lipids) in PBS. For the rhizobial inoculation, the rhizobia and labelled LDP1 were added to the liposome at the same time (in the final reaction also supplied with 1 mM Ca and 10 um Mn, the final rhizobial concentration is OD600=0.03).

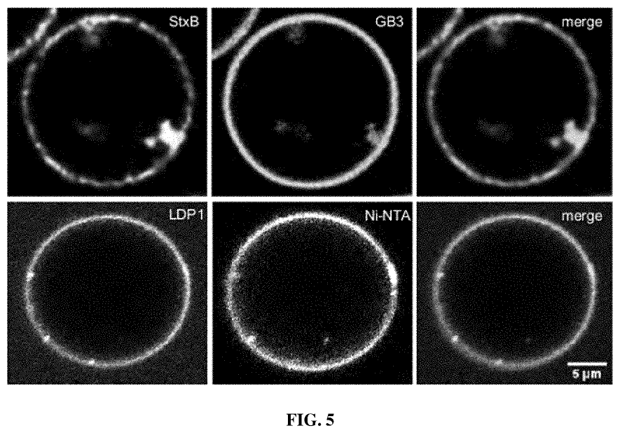

As shown in FIG. 5, addition of labelled LDP1 to liposomes resulted in LDP1 clustering and membrane invagination. In separate experiments, addition of rhizobia and labelled LDP1 to the liposome at the same time resulted in predominant LDP1 accumulation that coincided with rhizobia attachment sites on the liposome surface.

### Example 6: LDP1-Mediated Membrane Invaginations in Protoplasts in the Presence of Rhizobia

In order to investigate the ability of LDP1 to promote membrane invaginations in protoplasts in the presence of rhizobia, transgenic Agrobacterium tumefaciens carrying the plasmids of proLjUBI-GFP-LDP1 was infiltrated into Nicotiana benthamiana leaves together with the p19 silencing suppressor reaching a final concentration of OD600=0.3. Two days after infiltration, the transformed leaves were taken to isolate protoplasts. For rhizobial incubation, the overnight rhizobial liquid culture was centrifuged (3,000 rpm, 5 min) and the pellet re-suspended in PNT solution then add the rhizobia into the protoplast solution (with the final concentration is OD600=0.03). The protoplasts were incubated with rhizobia on a shaker (50 Mot/min) at room temperature for 3 hours (covered with aluminum foil) before taking the images. A control protoplast sample was incubated without adding rhizobia. The images were taken with a ZEISS light microscope with an Apotome.2 module and the software Imaris was used for further image analysis.

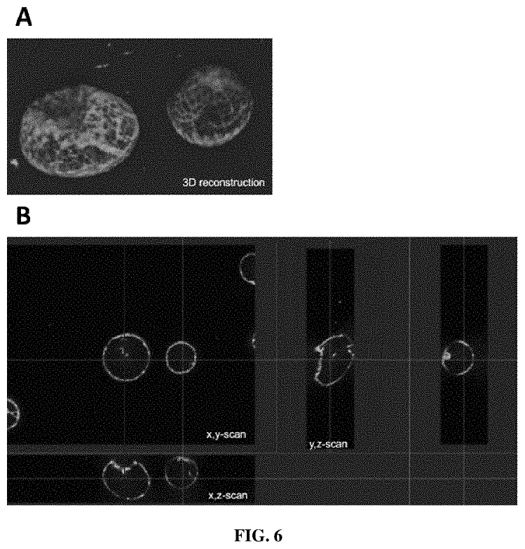

As shown in FIG. 6, N. benthamiana protoplasts ectopically expressing GFP-LDP1 exhibit membrane invaginations in the presence of rhizobia. FIG. 6A shows a 3D reconstruction of N. benthamiana protoplasts based on the scans shown in FIG. 6B.

### Example 7: LDP1 Associates With Polarly Secreted Rhizobial Polysaccharides

In order to investigate whether LDP1 directly associates with rhizobia, overnight grown rhizobial liquid cultures were centrifuged (3,000 rpm, 5 min) and the pellets were re-suspended in PBS solution (supplied with 1 mM Ca and 10 um Mn) and adjusted to OD600=0.1. Blocking was done using 3% BSA for 30 min at room temperature. Purified His-LDP1 protein was then added into the rhizobia solution (final concentration is 10m/ml) to incubate for another 45 min. Labelled rhizobia were washed three times with PBS solution (centrifuge at 1000 rpm for 30 secs to remove the solution). For immunofluorescence labelling, the first Anti-HIS antibody (Sigma-Aldrich) and secondary antibody (conjugated with Alex 488, Sigma-Aldrich) were all diluted in PBS solution supplemented with 3% BSA and each of them was incubated with rhizobia for 30 min. Before the incubation with the secondary antibody, the rhizobia were washed 3 times with PBS to remove the unbound Anti-HIS antibody. After incubating with the secondary antibody cells were washed 3 times before imaging. For the immuno-gold labeling, the secondary antibody was conjugated with gold (10 nm) instead of the Alex 488. After, 10 μl of the solution was dropped on the Grids for 10 min before methylcellulose treatment and negative stain.

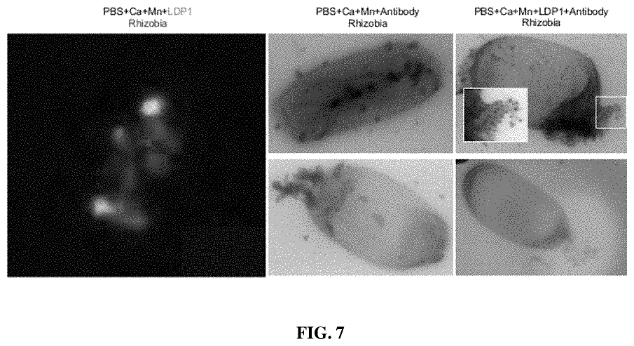

As shown in FIG. 7, recombinant LDP1 lectin domain can bind rhizobia in solution and polarly associates with a rhizobial exopolysaccharide matrix.

### Example 8: Membrane Stabilization by SYMREM1

Stabilization of membrane curvature and membrane invagination were further investigated. In these experiments, the properties of SYMREM1, a member of the plant-specific remorin family, were characterized. Electron microscopy (EM) and confocal laser-scanning microscopy were used to visualize and gain insights into the role of SYMREM1 during symbiotic infection.

Confocal laser-scanning microscopy images of protoplasts were obtained using a Leica TCS SP8 confocal microscope equipped with 20× water immersion lenses (Leica Microsystems, Mannheim, Germany) or a Zeiss LSM 880 with a 63× oil immersion lens. For HEK-293T cells, samples were fixed under green safe-light (520 nm). For this, the medium was replaced with 200 μl 4% (w/v) paraformaldehyde solution (PFA, Sigma-Aldrich, 1.00496). After 15 min incubation at room temperature, cells were washed twice with 500 μl DPBS. To stain cell nuclei, samples were incubated with 4′,6- diamidino-2-phenylindole (DAPI, Sigma-Aldrich D9542, 0.1 μg/ml) diluted in DPBS for 15 min, then washed twice with 500 μl DPBS before they were mounted on microscope slides in 8 μl mowiol mounting medium (2.4 g mowiol, 6 g glycerol, 6 ml H2O, 12 mL 0.2 M Tris/HCl, pH 8.5). Coverslips were fixed on the microscopy slides with nail polish. Images were acquired with a ZEISS LSM 880 laser scanning confocal microscope using a 63× Plan-Apochromat oil objective (NA 1.4). Z-stacks were acquired and images are shown in maximum intensity projection. For live cell imaging, cells were kept at 37° C. and 5% CO₂ in a stage top Tokai Hit incubator. Time series of Z-stacks were acquired after 3 cycles of Z-stacks (illumination 460 nm, 5 μmol m-2s-1 using a CoolLED pE-4000 universal illumination system). The 405 nm laser lines were used for DAPI, GFP was excited with a wild laser at 488 nm and the emission detected at 500-550 nm. YFP was excited with a 514 nm laser line and detected at 520-555 nm. mCherry fluorescence was excited at 561 nm and emission was detected between 575-630 nm. Samples, co-expressing two fluorophores were imaged in sequential mode between frames. All images analysis and projections were performed with either ImageJ/(Fiji) software (J. Schindelin et al., Nature Methods, 9, 676-682, 2012) or Imaris.

Transmission Electron Microscopy (TEM) was performed on nodules harvested at 3 wpi. Nodules were cut longitudinally in half, immediately fixed in MTSB (48) buffer containing 2.5% glutaraldehyde and 4% p-Formaldehyde under vacuum for 15 min (twice), and stored at 4° C. in fixative solution until further steps. After washing 5 times for 10 min each with buffer, nodules were post-fixed with 1% OsO4 in H₂O at 4° C. for 2h and again washed 5 times (10 min each) with H₂O at room temperature. The tissue was in block stained with 1% Uranyl Acetate for 1h in darkness, washed 3 times (10 min each) in H2O, and dehydrated in EtOH/H₂O graded series (30%, 50%, 70%, 80%, 90%, 95% 15 min each). Final dehydration was achieved by incubating the samples twice in absolute EtOH (30 min each) followed by incubation in dehydrated acetone twice (30 min each). Embedding of the samples was performed by gradually infiltrating them with Epoxy resin (Agar 100) mixed with acetone at 1:3, 1:1 and 3:1 ratio for 12 h each, and finally in pure Epoxy resin for 48h with resin changes every 12h. Polymerization was carried out at 60° C. for 48h. Ultrathin sections of approximately 70 nm were obtained with a Reichert-Jung ultra-microtome and collected in TEM slot grids. Images were acquired with a Philips CM 10 transmission electron microscope coupled to a Gatan BioScan 792 CCD camera at 80 kV acceleration voltage.

Scanning Electron Microscopy was carried out on freshly isolated protoplasts and on longitudinal vibratome sections (70m) of nodules collected after 3wpi. The material was immediately fixed, dehydrated as mentioned above (without Uranyl Acetate staining), and critical point dried in absolute EtOH-CO₂. Dried material was mounted on carbon tabs and coated with platinum at 5 nm. Imaging of samples was performed using a Hitachi S-4800 microscope.

Negative staining of purified SYMREM1 protein was performed by applying 5 μl protein solution to glow-discharged 400 Cu mesh carbon grids for 10 min, blotting and negatively staining using 2% (w/v) uranyl acetate. Images were recorded under low-dose conditions on a Talos F200C transmission electron microscope operated at 200 kV and equipped with a Ceta 16M camera. Micrographs were taken at a nominal magnification of 73,000×. A total of 389 segments were manually selected using RELION-3.1.0 (J. Zivanov et al., eLife, 7, e42166, 2018). The defocus and astigmatism of the images were determined with CTFFIND4.1 (A. Rohou and N. Grigorieff, Journal of Structural Biology, 192, 216-221, 2015) and numerical phase-flipping was done to correct for effects of the contrast transfer function using RELION-3.1.0. Image processing was done using IMAGIC-5 (M. van Heel et al., Journal of Structural Biology, 116, 17-24, 1996). Particle images were band pass filtered between 400 and 10 Å, normalized and centered by iteratively aligning them to a vertically oriented class average. Class averages containing 5-10 images were obtained by four rounds of classification based on multivariate statistical analysis, followed by multi-reference alignment using homogenous classes as new references.

### Example 9: SYMREM1 Forms Liquid-Liquid Phase Separations (LLPS)

SYMREM1 displays an intrinsically disordered (IDR)N-terminal and a conserved C-terminal coiled-coil region. Such long and conformationally heterogeneous IDRs can adopt secondary structures under certain physiological conditions. In order to assess the general ability of SYMREM1 to form LLPS (Liquid-liquid phase separation), an established optogenetic setup frequently used for human proteins was used (N. Schneider et al., Science advances, 7, 2021), for which a light-sensitive Cry2 interaction domain was N-terminally fused to fluorescently tagged mCherry-SYMREM1 in order to generate photo-switchable oligomers. Plasmids used in the LLPS assays were created via AQUA cloning and are based on a pEGFP-C3 backbone. Specifically, Human embryonic kidney cells (HEK-293T, DSMZ, ACC 305) were cultivated at 37° C. and 5% CO2 in DMEM complete medium (DMEM (PAN Biotech, P04-03550) supplemented with 10% (v/v) fetal calf serum (FCS, PAN Biotech, P30-3306), 100 U ml-1 penicillin and 100 μg ml-1 streptomycin (PAN Biotech, P06-07100)). Cells were passaged every 2-3 days upon reaching≈90% confluency. For microscopy, high precision cover slips (Roth, LH23.1) were placed into empty wells of 24-well plates (Corning, 3524). To enhance adherence of the cells, coverslips were coated with 500 μl 20 μg/ml rat tail collagen I (Thermo Fisher, A1048301) diluted in 25 mM acetic acid. After 1 h incubation at room temperature, coverslips were washed twice with 500 μl DPBS (2.7 mM KCl, 1.5 mM KH2PO4, 8.1 mM Na2HPO4, 137 mM NaCl) and 70,000 cells were seeded in 500 μl DMEM complete medium per well. 24 h later, polyethyleneimine (PEI, Polyscience, linear, MW: 25 kDa) transfection was performed. To this aim, the transfection mix consisting of 200 ng DNA and 0.66 μL PEI (1 mg/ml) in 50 μl OptiMEM (Thermo Fisher, 22600134) per well, was mixed thoroughly and incubated for 15 min at room temperature before dropwise addition to the cells. Experiments were started 24 h later. For live cell imaging 350,000 cells were seeded on 35 mm μ-Dishes (Ibidi, 81156) in 2 mL DMEM complete medium. For transfection, 1 μg DNA and 3.3 μL PEI (1 mg/ml) in 250 μl OptiMEM was used.

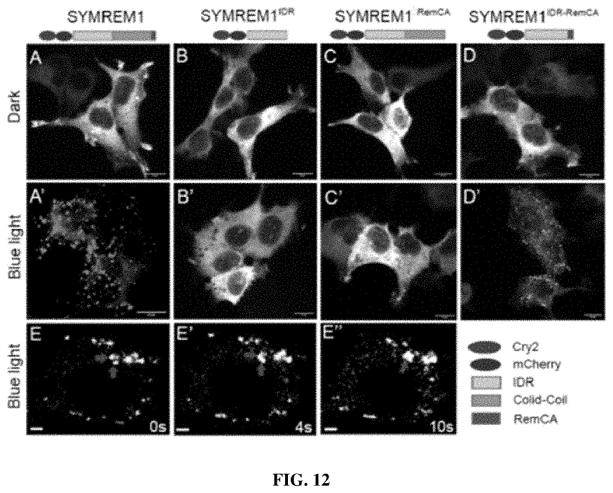

Expression of full-length SYMREM1 in dark-exposed HEK-293T cells revealed a rather homogenous distribution in the cytosol while blue-light (465 nm) induced oligomerization resulted in LLPS-like opto-condensate formation (FIG. 12A-12A′). Neither expression of the SYMREM1 IDR (SYMREM1^{IDR}) nor a variant truncated by the C-terminal remorin anchor (RemCA) peptide (SYMREM1^{ΔRemCA}) resulted in LLPS while an IDR-RemCA fusion (SYMREM1^{IDR-RemCA}) can restore these phase-separated condensates (FIG. 12B-12D′). Furthermore, these condensates fused over time (FIG. 12E-12E″), a hallmark of LLPS. These data indicate that SYMREM1 has the general ability to form LLPS. Crowding in LLPS at high protein concentrations may also result in an auto-assembly into higher order filamentous structures as shown herein.

### Example 10: SYMREM1 Auto-Assembly into Higher Order Filamentous Structures and Impact on Membrane Tubulation

In order to investigate whether SYMREM1 can auto-assemble into higher order filamentous structures, transmission electron microscopy (TEM) and 2D classification was performed on purified, recombinant SYMREM1. To obtain purified SYMREM1, the SYMREM1 protein coding sequence of Medicago truncatula was recombined into the Gateway (GW) compatible pDEST17 vector via LR-reaction. E. coli BL21(DE3) cells were transformed with plasmid pDEST17 encoding His-SYMREM1 protein. A single colony of transformed E. coli was transferred in LB media and grown overnight to make pre-culture. Then, 40 ml of pre-culture was inoculated in 2L of LB media and culture was grown at 37° C. Protein expression was induced by 1 mM IPTG at OD600 of 0.6. Afterwards, cells were incubated overnight (about 20 h) at 25° C. Cells were harvested by centrifugation at 6000 g for 15 min. The cell pellet was resuspended in 100 ml Lysis buffer (20 mM HEPES, 500 mM NaCl, 20 mM imidazole, 10% glycerol, 1 mM EDTA, 1 mM Pefabloc, pH 7.2) and cells were passed through Constant Cell Disrupter (Constant Systems Limited). Cell debris was removed by centrifugation at 30,000 g for 30 min. The cleared cell lysate was loaded onto IMAC column (5 ml HisTrap_FF) pre-equilibrated with Loading buffer A (20 mM HEPES, 500 mM NaCl, 20 mM imidazole, pH 7.2) and washed with 10 CV of Loading buffer A. Proteins were eluted with a linear gradient of imidazole from 20 to 450 mM in 15 CV. The eluted fractions were pooled and concentrated by spin filtration to 5 ml. Precipitated proteins were removed by an additional centrifugation for 10 min at 10,000 g before loading onto gel-filtration column (HiLoad Superdex 200 16/60) equilibrated with PBS. Eluted fractions after gel-filtration were analyzed with SDS-PAGE, those containing a pure His-SYMREM1 were pooled and concentrated by spin filtration to the working concentration.

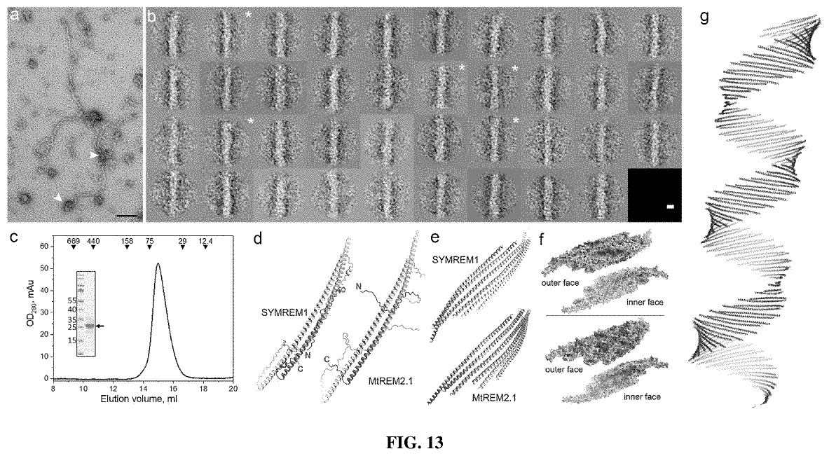

Auto-assembling and amorphous protein filaments that were partially branched or scrambled were detected (FIG. 13A). Systematic inspection of 389 particles revealed an average width between 84 and 125 Å for the filamentous particles with some of them showing a helical conformation (FIG. 13B). Irregular protein bodies (FIG. 13A) that may represent filament seeds were also observed. Since the filaments were too amorphous for further structural assessment by cryo-EM, we conducted ab initio modelling considering trimeric core units as a basis. The full-length SYMREM1 protein with its flexible N-terminal IDR as well as an antiparallel alignment of the monomers were considered in this example. Structural features of SYMREM1 monomers were modelled using the I-TASSER server (Iterative Threading ASSEmbly Refinement, Zhang, et al., Nature Protocols 5:725-738, 2010) and the model with the highest c-score was used to compute the SYMREM1 trimer on the PATCHDOCK server (Schneidman-Duhovny, et al., Nucl. Acids. Res. 33: W363-367, 2005).

This modelling predicted banana-shaped units spanning a 140 Å space over their concave site with an over-representation of positively charged patches in this region (FIG. 13C-13E). Additional 3D image fitting and super-exposition revealed similarities with human endophilin A1 (FIG. 13D-13D″), a N-BAR-domain protein that functions in membrane bending (M. R. Ambroso et al., Proceedings of the National Academy of Science, 111, 6982-6987, 2014). Besides inducing local membrane curvature, several BAR proteins have been shown to drive and stabilize inwards and outwards directed membrane tubulation (Bhatt et al., Structure, 29, 61-69.e69, 2021). These results support a putative function of SYMREM1 in membrane morpho-dynamics and stabilization of membrane curvatures.

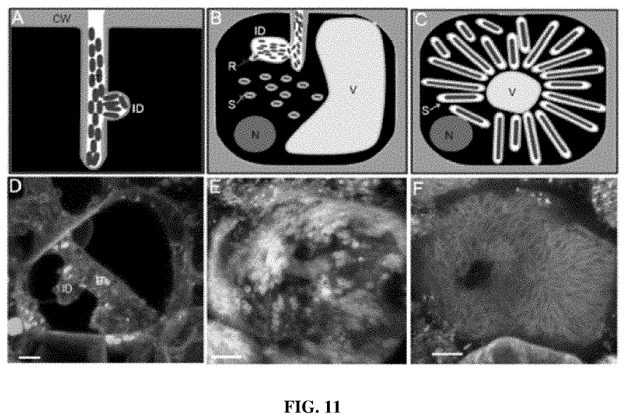

To further asses the function of SYMREM1 in membrane morpho-dynamics and stabilization of membrane curvatures, SYMREM1-positive symbiotic membranes such as ITs, IT droplets, and bacterial release sites were visualized at sub-cellular resolution. To visualize these symbiotic membranes, phosphatidylserine (PS), a central phospholipid of biological membranes, was labelled by expressing a LactC2 biosensor that allowed clear imaging of membrane contours at the three selected target sites (FIG. 11A-11F). For analyzing the subcellular localization of SYMREM1 and the LactC2 biosensor in WT and ipd3, the corresponding constructs were used to transform Medicago plants by hairy root transformation and visualized using confocal microscopy.

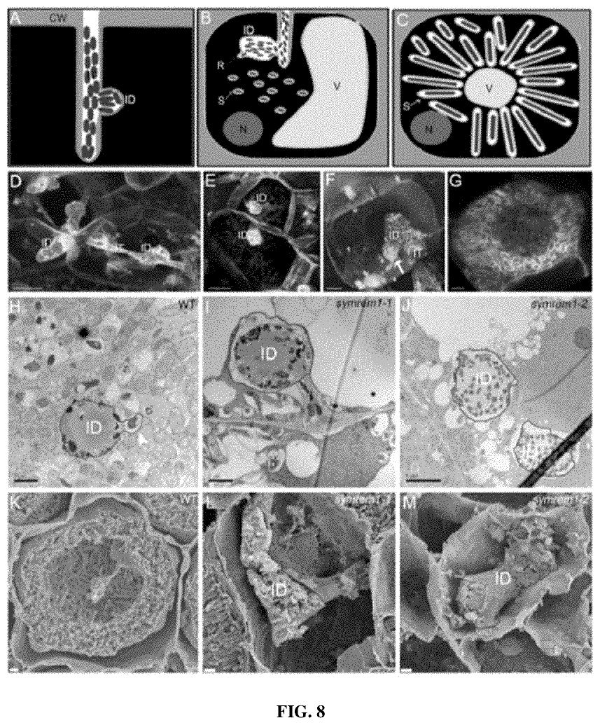

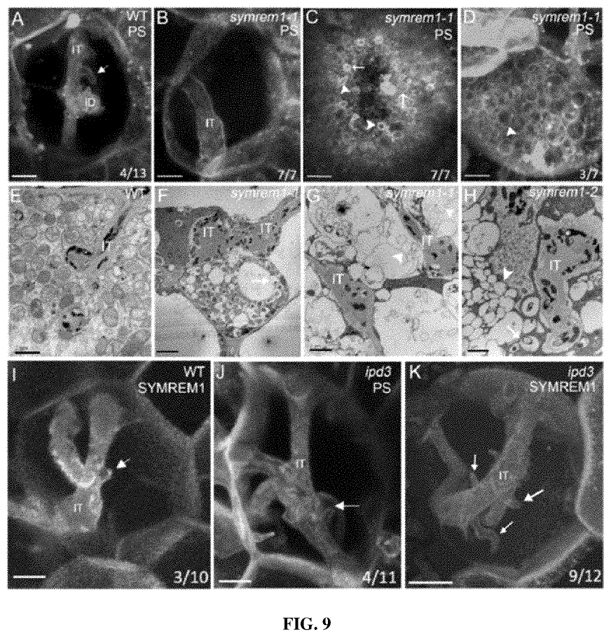

Detailed inspection of these structures revealed numerous membrane protrusions associated with growing or bacteria-releasing ITs in wild-type (WT) Medicago truncatula plants (FIG. 9A). These latter structures preceded the intracellular release of bacteria into plant cells prior to the onset of symbiotic nitrogen fixation and likely provide size-restricted temporal membrane reservoirs. The occurrence of these membrane tubes coincided with IT droplets and bacterial release sites, structures showing predominant SYMREM1 protein accumulations (FIG. 8A-8C). In line with this and in contrast to wild-type plants, symrem1 mutants failed to release bacteria in a majority of cells in the inner nodule cortex and exhibited bulky ITs, as revealed by light and electron microscopy (EM) (FIG. 8H-8M). These ITs, however, did not display stabilized tubulation (FIG. 9B). Instead, large numbers of detached empty membrane spheres were observed in IT-containing but release-deficient nodule cortex cells using confocal laser-scanning microscopy (FIGS. 9C and 9D) and TEM (FIG. 9E-9H). This is in sharp contrast to WT plants, where bacterial differentiation and symbiosome formation occurred normally with the symbiosome membrane being tightly associated with the differentiated bacteroids (FIGS. 8G and 9E).

To test the impact of SYMREM1 on membrane tubulation in vivo, fluorescently-labelled SYMREM1 was ectopically expressed in wild-type M. truncatula plants. However, the frequency of stabilized membrane tubulation remained unaltered compared to that observed in LactC2-labelled WT cells (FIG. 91). As this was likely due to the high membrane turnover of these structures at symbiotically active membrane interfaces, the release-compromised ipd3-1 mutant was used, which is defective in the transcriptional activator CYCLOPS that regulates, among other genes, the expression of endogenous SYMREM1 (Horvath, et al., Molecular plant microbe interactions 1345-1358 (2011)). In these mutants, few membrane tubes were found in 36% of all observed cases (FIG. 9J) whereas ectopic expression of fluorophore-tagged SYMREM1 in this genetic background significantly increased IT-associated membrane tubulation to 75% with several tubes per IT being observed frequently (FIG. 9K). These data further support a function of SYMREM1 in membrane tubulation.

### Example 11: SYMREM1 Stabilizes Membrane Curvatures

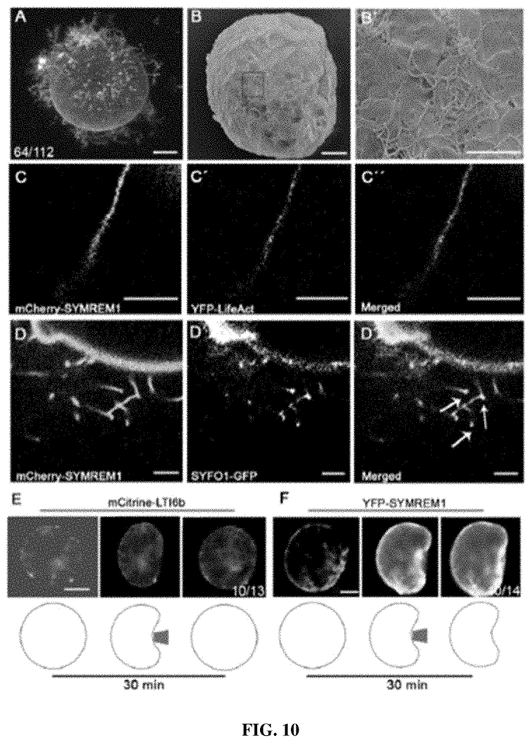

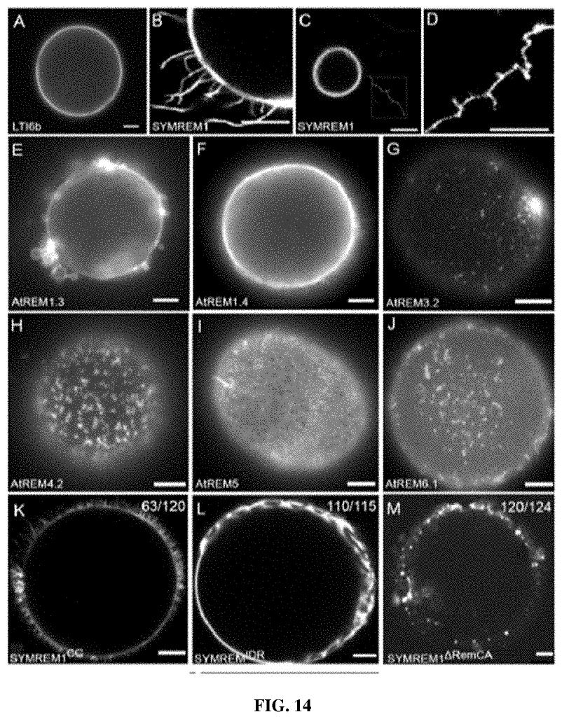

In order to test whether membrane curvature can be altered by SYMREM1 in the absence of a cell wall, SYMREM1 was expressed in cell wall-devoid Nicotiana benthamiana protoplasts that are naturally devoid of SYMREM1. When isolating mesophyll protoplasts ectopically expressing SYMREM1, numerous tubular outgrowths were observed developing shortly after protoplasting the tissue with an average width of 178±0.03 nm as assessed by scanning EM. This phenomenon was observed on 64 out of 112 (57%) inspected protoplasts (FIG. 10A-10B′, S4B-D) while membrane tubes on control protoplasts were not retrieved (FIG. 14A). This molecular ability seems conserved within the remorin protein family since membrane blebbing or various degrees of tubulation were observed when expressing different Arabidopsis remorins in protoplasts (FIG. 14E-J).

To further dissect the protein domain responsible for this effect, further protein variants were generated and tested. Similar rates of membrane tubulation were found upon expression of the isolated coiled-coil domain (SYMREM1^{CC}; FIG. 14K) but not when expressing the isolated IDR (SYMREM1^{IDR}; FIG. 14L) or a protein variant lacking the C-terminal anchor peptide (SYMREM1^{ΔRemCA}; FIG. 14M). These latter two constructs were cytosolic and only induced mild membrane ruffling (FIG. 14L-M).

Since long and tip-growing plant membrane protrusions such roots hairs and pollen tubes as well as filopodia of human cells comprise central actin elements, the presence and polar assembly of actin was assessed by co-expressing the actin marker Lifeact and the symbiotic formin SYFO1 (P. Liang et al., Current Biology, 31, 2712-2719.e2715, 2021), respectively, with SYMREM1 in protoplasts. Indeed, all tubes contained a central actin filament that co-localized with SYMREM1 (FIG. 10C-10C″) and tip-localized SYFO1 (FIG. 10D-10D″). Since SYFO1 can induce initial membrane protrusions on protoplasts and formin condensation relies on membrane surface scaffolding, SYMREM1 might stabilize rather than actively drive these membrane tubulations. To test whether SYMREM1 exclusively functions as a stabilizing scaffold for unidirectional curvature, maintenance of negative curvatures in the presence of this protein were assessed as an indication of underlying IT-associated membrane tubes. SYMREM1-expressing protoplasts were isolated and immediately indented with a micro-capillary for 30 minutes. In particular, isolated protoplasts were embedded in 0.5% agarose on a cover of a Petri Dish. The injection set-up consisted of an inverted microscope (Zeiss Axiovert 135 TV) with a motor driven micromanipulator (LANG GmbH & Co. KG, Type: STM3) mounted at the right side of the stage. Femtotips injection needles (Eppendorf) were adapted by removing the sharp-pointed tip of the needle by hand, until obtaining a needle that could not penetrate the protoplast plasma-membrane.

In these experiments, 10/13 protoplasts expressing an mCitrine-LTI6b membrane marker (control) fully re-inflated immediately after releasing the pressure (FIG. 10E). By contrast, only 4/14 protoplasts expressing SYMREM1 re-inflated, while microcapillary-induced membrane deformations were maintained in 10/14 of these protoplasts (FIG. 10F). These data show that SYMREM1 is able to generally stabilize plant membrane curvatures in a symbiotic context.

### Example 12: Enhanced Symbiotic Infection in Transgenic Non-Legume Plants Overexpressing LDP1

Recombinant constructs comprising a nucleotide sequence encoding the LDP1 protein are transformed into Solanum lycopersicum plant cells, which are regenerated to produce transgenic plants overexpressing LDP1 protein. Plants are inoculated with rhizobia and evaluated phenotypically as described herein for morphological changes related to symbiotic infection, including but not limited to membrane invaginations, local clustering of rhizobia at the membrane, focal arrangement of the actin and the microtubule cytoskeleton.

### Example 13: Enhanced Symbiotic Infection in Protoplasts and Transgenic Plants Co-Expressing LDP1 and SYMREM1

A recombinant construct comprising a nucleotide sequence encoding the LDP1 protein and a recombinant construct comprising the SYMREM1 protein are transformed into protoplasts; and Medicago truncatula plant cells; which are regenerated to produce transgenic plants overexpressing LDP1 and SYMREM1 proteins. Transgenic protoplasts and plants are inoculated with rhizobia and evaluated phenotypically as described herein for morphological changes related to enhanced symbiotic infection, including increased nodule number per plant and increased infection threads per plant. Plants overexpressing LDP1 and SYMREM1 proteins exhibit enhanced symbiotic infection compared with plants not comprising the recombinant LDP1 and SYMREM1 constructs.

### Example 14: Enhanced Performance in Transgenic Plants Co-Expressing LDP1 and SYMREM1 Under Nitrogen-Limited Conditions

A recombinant construct comprising a nucleotide sequence encoding the LDP1 protein and a recombinant construct comprising the SYMREM1 protein are transformed into Medicago truncatula plant cells, which are regenerated to produce transgenic plants overexpressing LDP1 and SYMREM1 proteins. Plants are grown under nitrogen-limited conditions and inoculated with rhizobia. Plants are evaluated phenotypically as described herein for enhanced symbiotic infection, including increased nodule number per plant and increased infection threads per plant, as well as increased biomass, yield, and seed production. Plants overexpressing LDP1 and SYMREM1 proteins exhibit enhanced symbiotic infection and agronomic performance compared with plants not comprising the recombinant LDP1 and SYMREM1 constructs under nitrogen-limited conditions.

### Example 15: Activation of the LDP1 Promoter in Tomato Roots by Inoculation with AM Fungi

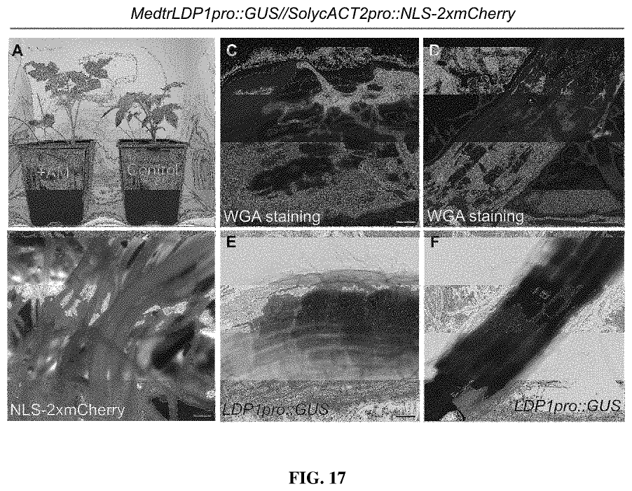

Six-week old tomato hairy roots (Solanum lycopersicum cv. M82 WT*) were transformed with Agrobacterium rhizogenes strain Arqua1 carrying a MedtrLDP1pro::GUS:t35S//SolycACT2pro::NLS-2xmCherry:t35S (proMedtrLDP1pro::GUS:t35S//proSolycACT2::NLS-2xmCherry:t35S) construct. Tomato plants were transferred to vermiculite/sand pots containing arbuscular mycorrhiza fungi for two weeks. Roots were stained with X-gluc staining solution to observe GUS activity and AM structures were stained with WGA-Alexa Fluor 488. FIG. 17 shows activation of the LDP1 promoter in tomato hairy roots inoculated with AM fungi.

### Example 16: Nodule-like Structure Formation in Tomato and Tobacco Hairy Roots Overexpressing NFP/LYK3

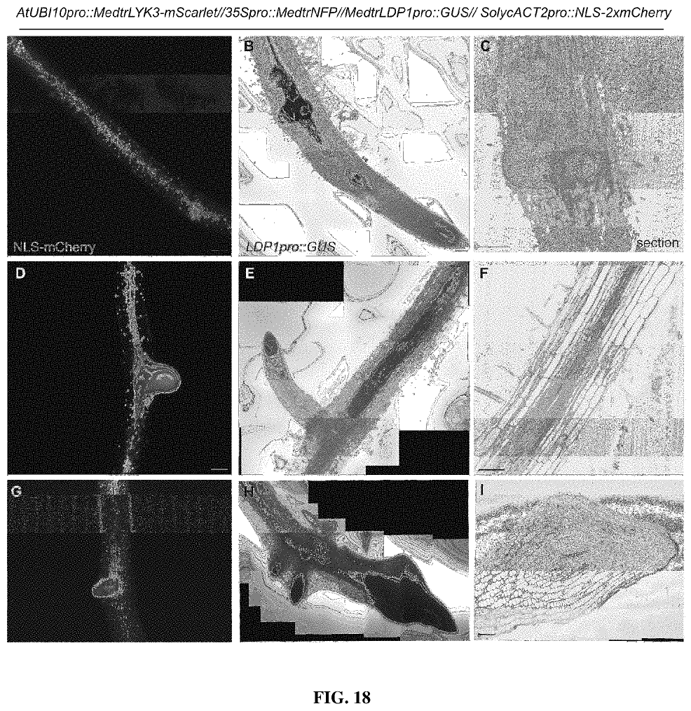

Six-week-old tomato hairy roots (Solanum lycopersicum cv. Moneymaker) were transformed with Agrobacterium Rhizogenes strain Arqua1 carrying Medicago NFP/LYK3 (proAtUB 110::MedtrLYK3-mScarlet:t35S//pro35So::MedtrNFP:t35S//proSolycACT2::NLS-2xmCherry:t35S) receptor constructs. Transformed tomato plants were transferred to vermiculite/sand pots and inoculated with S. meliloti (FIG. 18A-C), an S. meliloti and rhizobium mixture (FIG. 18 D, E) or both (FIG. 18 G-I) (OD ˜0.3) for 7 days. Transformed roots were selected (FIGS. 18A, D and G). To observe GUS activity, transgenic roots were stained with X-gluc buffer (FIGS. B, E, and H) and 9-10 μm longitudinal sections of GUS-stained roots were further stained for 15 min in 0.1% Ruthenium Red (FIGS. 18 C, F, and I).

Inoculation of tomato hairy roots with S. meliloti, a mixture of S. meliloti and rhizobium, or both, induced the LDP1 promoter and nodule-like structures in tomato hairy roots overexpressing NFP/LYK3 (using a proAtUB 110::MedtrLYK3-mScarlet:t35S//pro35S:: MedtrNFP:t35S//proMedtrLDP1::GUS:t35S//proSolycACT2::NLS-2xmCherry:t35T construct) (FIG. 18).

Similar results were observed in tobacco hairy roots overexpressing NFP/LYK3 (using a proAtUBI10::MedtrLYK3-mScarlet:t35S//pro35S::MedtrNFP:t35S//proMedtrLDP1::GUS:t35S//proSolycACT2::NLS-2xmCherry:t35T construct) and inoculated with rhizobia.

### Example 17: Expression of AtREM3.2 (At4g00670) in Tobacco Leaf Epidermal Cells Mediates Stabilization of Membrane Topologies

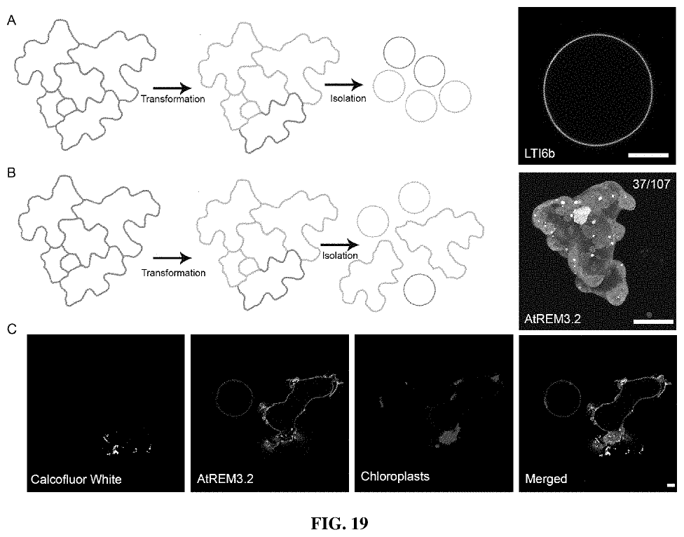

In order to investigate expression of AtREM3.2 in tobacco leaf epidermal cells, leaves were first infiltrated with Agrobacteria and then allowed to express the AtREM3.2 construct (proLjUBI::GFP-AtREM3.2:t35S). Protoplasts were isolated from these leaves resulted in a mixed population with high number of cells maintaining the jigsaw puzzle shape (FIG. 19). These experiments demonstrate that expression of AtREM3.2 (At4g00670) in tobacco leaf epidermal cells efficiently mediates stabilization of membrane topologies.

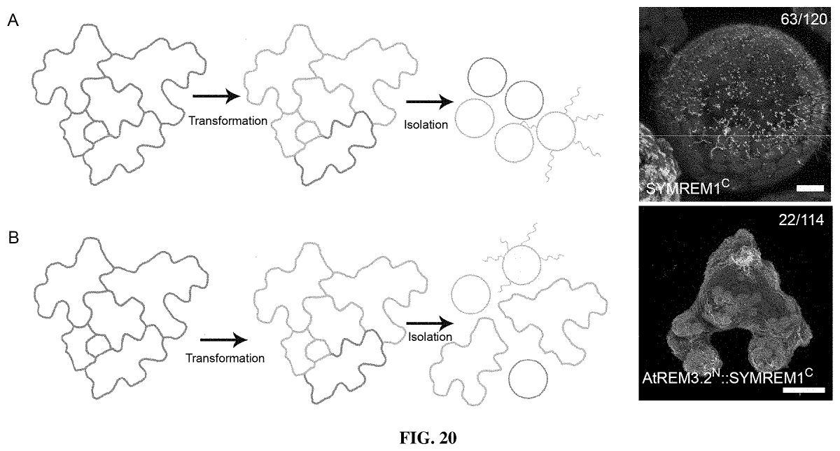

Expression of an AtREM3.2^{N}/SYMREM1^{C} (proLjUBI::GFP-AtREM3.2^{N}/SYMREM1^{C}: t35S) chimera construct in tobacco leaf cells was also investigated. Leaves were first infiltrated with Agrobacteria and then allowed to express the chimeric AtREM3.2/MtSYMREM1 construct. Isolation of protoplasts from these leaves resulted in a mixed population with high number of cells maintaining the jigsaw puzzle shape (FIG. 20) These results demonstrate that expression of the AtREM3.2^{N}/MtSYMREM1^{C} chimera efficiently mediates stabilization of membrane topologies.

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

What is claimed is:

1. A recombinant DNA molecule comprising a heterologous promoter operably linked to a polynucleotide segment encoding a lectin-domain containing protein or fragment thereof, wherein:
a. said protein comprises the amino acid sequence of any of SEQ ID NOs: 2 and 4-12;
b. said protein comprises an amino acid sequence having at least 85%, or 90%, or 95%, or 98% or 99%, or about 100% amino acid sequence identity to any of SEQ ID NOs: 2 and 4-12; or
c. said polynucleotide segment hybridizes under stringent hybridization conditions to a polynucleotide having the nucleotide sequence of SEQ ID NO: 1.

2. The recombinant DNA molecule of claim 1, wherein:
a. said recombinant DNA molecule is expressed in a plant cell to produce an increase in symbiotic infection of a bacteria or fungi; or
b. said recombinant DNA molecule is in operable linkage with a vector, and said vector is selected from the group consisting of a plasmid, phagemid, bacmid, cosmid, and a bacterial or yeast artificial chromosome.

3. The recombinant DNA molecule of claim 1, present within a host cell, wherein said host cell is selected from the group consisting of a bacterial cell and a plant cell.

4. The recombinant DNA molecule of claim 3, wherein said bacterial host cell is from a genus of bacteria selected from the group consisting of: Agrobacterium, Rhizobium, Bacillus, Brevibacillus, Escherichia, Pseudomonas, Klebsiella, Pantoea, and Erwinia.

5. The recombinant DNA molecule of claim 4, wherein said Bacillus is Bacillus cereus or Bacillus thuringiensis, said Brevibacillus is a Brevibacillus laterosperous, and said Escherichia is a Escherichia coli.

6. The recombinant DNA molecule of claim 3, wherein said plant cell is a dicotyledonous or a monocotyledonous plant cell.

7. The recombinant DNA molecule of claim 6, wherein said plant cell is selected from the group consisting of an alfalfa, banana, barley, bean, broccoli, cabbage, brassica, canola, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, watermelon, and wheat plant cell.

8. A plant or part thereof comprising the recombinant DNA molecule of claim 1.

9. The plant or part thereof of claim 8, further comprising a recombinant DNA molecule comprising a heterologous promoter operably linked to a polynucleotide segment encoding a protein or fragment thereof, wherein:
a. said protein comprises the amino acid sequence of any of SEQ ID NOs: 14 and 16-19;
b. said protein comprises an amino acid sequence having at least 85%, or 90%, or 95%, or 98% or 99%, or about 100% amino acid sequence identity to any of SEQ ID NOs: 14 and 16-19; or
c. said polynucleotide segment hybridizes under stringent hybridization conditions to a polynucleotide having the nucleotide sequence of SEQ ID NO: 13.

10. The plant or part thereof of claim 8, wherein said plant is a monocot plant or a dicot plant.

11. The plant or part thereof of claim 10, wherein said plant is selected from the group consisting of an alfalfa, banana, barley, bean, broccoli, cabbage, brassica, canola, carrot, cassava, castor, cauliflower, celery, chickpea, Chinese cabbage, citrus, coconut, coffee, corn, clover, cotton, a cucurbit, cucumber, Douglas fir, eggplant, eucalyptus, flax, garlic, grape, hops, leek, lettuce, Loblolly pine, millets, melons, nut, oat, olive, onion, ornamental, palm, pasture grass, pea, peanut, pepper, pigeon pea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, watermelon, and wheat.

12. A transgenic seed comprising the recombinant DNA molecule of claim 1.

13. A method of producing progeny seed comprising the recombinant DNA molecule of claim 1, the method comprising:
a. planting a first seed comprising the recombinant DNA molecule of claim 1;
b. growing a plant from the seed of step a; and
c. harvesting the progeny seed from the plants, wherein said harvested seed comprises said recombinant DNA molecule.

14. A plant susceptible to symbiotic infection, wherein the cells of said plant comprise the recombinant DNA molecule of claim 1.

15. A method for increasing symbiotic infection in a plant, said method comprising:
a. expressing a lectin-domain containing protein or fragment thereof as set forth in any of SEQ ID NOs: 2 and 4-12 in a plant;
b. contacting said plant with an effective amount of one or more rhizobia bacterium, arbuscular mycorrhiza fungi, or a combination thereof.

16. The method of claim 15, wherein:
a. said rhizobia bacterium is selected from the group consisting of: S. meliloti, Mesorhizobium loti, Sinorhizobium meliloti, Sinorhizobium fredii, Rhizobium sp. IRBG74 and NGR234, Bradyrhizobium sp.; or
b. said arbuscular mycorrhiza fungi is selected from the group consisting of: R. irregularis, Rhizophagus intraradices, Glomus mosseae, and Funneliformis mosseae.

17. The method of claim 15 further comprising expressing a protein or fragment thereof as set forth in any of SEQ ID NO: 14 and 16-19 in the plant.

18. A recombinant DNA molecule comprising a polynucleotide sequence selected from the group consisting of:
a) a sequence with at least 85 percent sequence identity to any of SEQ ID NO: 3;
b) a sequence comprising SEQ ID NO: 3; and
c) a fragment of SEQ ID NO: 3, wherein the fragment has gene-regulatory activity;
wherein said sequence is operably linked to a heterologous transcribable polynucleotide molecule.

19. The recombinant DNA molecule of claim 18, wherein the sequence provides expression of said heterologous transcribable polynucleotide molecule in response to an external stimulus.

20. The recombinant DNA molecule of claim 18, wherein the sequence provides expression of said heterologous transcribable polynucleotide molecule in a root hair cell.

21. A transgenic plant cell comprising the recombinant DNA molecule of claim 18.

22. A transgenic plant, plant part, or seed, comprising the recombinant DNA molecule of claim 18.

23. A progeny plant of the transgenic plant of claim 22, or a part thereof, wherein the progeny plant or part thereof comprises said recombinant DNA molecule.

24. A method of expressing a transcribable polynucleotide molecule comprising obtaining a transgenic plant according to claim 22 and cultivating plant, wherein the transcribable polynucleotide is expressed.

25. The plant or part thereof of claim 8, further comprising a recombinant DNA molecule comprising a heterologous promoter operably linked to a polynucleotide segment encoding a protein or fragment thereof, wherein:
a. said protein comprises the amino acid sequence of SEQ ID NO: 21 or SEQ ID NO: 23;
b. said protein comprises an amino acid sequence having at least 85%, or 90%, or 95%, or 98% or 99%, or about 100% amino acid sequence identity to SEQ ID NO: 21 or SEQ ID NO: 23; or
c. said polynucleotide segment hybridizes under stringent hybridization conditions to a polynucleotide having the nucleotide sequence of SEQ ID NO: 20 or SEQ ID NO: 22.

26. The plant or part thereof of claim 25, further comprising a recombinant DNA molecule comprising a heterologous promoter operably linked to a polynucleotide segment encoding a protein or fragment thereof, wherein:
a. said protein comprises the amino acid sequence of any of SEQ ID NOs: 14 and 16-19;
b. said protein comprises an amino acid sequence having at least 85%, or 90%, or 95%, or 98% or 99%, or about 100% amino acid sequence identity to any of SEQ ID NOs: 14 and 16-19; or
c. said polynucleotide segment hybridizes under stringent hybridization conditions to a polynucleotide having the nucleotide sequence of SEQ ID NO: 13.

27. The plant or part thereof of claim 8, further comprising a recombinant DNA molecule comprising a heterologous promoter operably linked to a polynucleotide segment encoding a protein or fragment thereof, wherein:
a. said protein comprises the amino acid sequence of SEQ ID NO: 25;
b. said protein comprises an amino acid sequence having at least 85%, or 90%, or 95%, or 98% or 99%, or about 100% amino acid sequence identity to SEQ ID NO: 25; or
c. said polynucleotide segment hybridizes under stringent hybridization conditions to a polynucleotide having the nucleotide sequence of SEQ ID NO: 24.

28. The plant or part thereof of claim 27, further comprising a recombinant DNA molecule comprising a heterologous promoter operably linked to a polynucleotide segment encoding a protein or fragment thereof, wherein:
a. said protein comprises the amino acid sequence of any of SEQ ID NOs: 14 and 16-19;
b. said protein comprises an amino acid sequence having at least 85%, or 90%, or 95%, or 98% or 99%, or about 100% amino acid sequence identity to any of SEQ ID NOs: 14 and 16-19; or
c. said polynucleotide segment hybridizes under stringent hybridization conditions to a polynucleotide having the nucleotide sequence of SEQ ID NO: 13.
